(19) United States
(12) Patent Application Publication
(10) Pub. No.: US 2018/0303744 A1
(43) Pub. Date: Oct. 25, 2018

(54) COSMETIC COMPOSITION COMPRISING POLYURETHANE

(71) Applicant: Covestro Deutschland AG, Leverkusen (DE)

(72) Inventors: Sebastian DÖRR, Düsseldorf (DE); Sophie VIALA, Köln (DE); Xiaohui XIONG, Shanghai (CN)

(21) Appl. No.: 15/771,586

(22) PCT Filed: Oct. 30, 2015
(86) PCT No.: PCT/CN2015/093327

§ 371 Date: Apr. 27, 2018

(51) Int. Cl. A61K 8/87; A61Q 1/02; A61Q 1/10; A61Q 3/02; A61Q 5/06; A61Q 19/00; A61Q 19/08; A61Q 17/04

(52) U.S. Cl. CPC A61K 8/87; A61Q 1/02; A61Q 1/10; A61Q 17/04; A61Q 5/06; A61Q 19/00; A61Q 19/08; A61Q 3/02

(57) ABSTRACT

A cosmetic composition comprises at least one water-dispersible polyurethane polymer obtainable by the reaction of at least one isocyanate-functional polyurethane pre-polymer A) with at least one isocyanate-reactive component B), wherein the at least one isocyanate-functional polyurethane pre-polymer A) is obtainable by the reaction of at least one polyol component A1), and at least one polyisocyanate component A2) comprising 60 wt % or more, based on the total weight of polyisocyanates in the pre-polymer formation reaction, of at least one saturated cycloaliphatic polyisocyanate, and wherein the molar ratio of the isocyanate groups to the isocyanate-reactive groups in the pre-polymer A) formation reaction is at least 1.90. The composition provides improved durability and holding power under high humidity conditions and reduced flake formation during combing.

The present invention relates to a cosmetic composition comprising a specific water-dispersible polyurethane, the use of such a cosmetic composition for the application on keratinous fibers, on human skin, on human mucous membranes and on nails, in particular, for the treatment of human hair, the use of a specific polyurethane polymer for the manufacture of a cosmetic composition, and a method of a cosmetic treatment of a human being, which involves in particular the topical application of the cosmetic composition of the invention.

### RELEVANT STATE OF THE ART

Cosmetic compositions comprising polyurethanes are known in the prior art. For example more recent patent publications include WO 2009/118105 A1, WO 2012/130683 and U.S. Pat. No. 7,445,770 B2 which relate to hair setting compositions comprising specific polyurethanes. WO 2009/118106 A1 relates to decorative cosmetic compositions comprising specific polyurethanes or aqueous dispersions thereof. WO 2015/075280 A1 relates to a hair-styling composition, containing solvent based polyurethane urea solutions. Polyurethanes for the manufacture of waterproof breathable coatings comprising dicyclohexylmethane diisocyanate are disclosed in WO 2013/037767 A2.

### Technical Problem Solved

Current hair styling trends reveal consumer desire to combine high performance, e.g. durability, and strong hold, with natural feel and look, e.g. flexible style and soft touch, in an ideal styling product. In fact, the expectations of consumer of a styling product are set high: strong holding power, durable styling/fixative effect even after mechanical action, excellent curl retention at high humidity, natural hair style, smooth feel and lustrous healthy appearance (Sophie Viala, Yuliya Berezkin, “Polyurethane dispersions keep hair in shape”, Focus on HAIR CARE-Supplement to Household and Personal Care TODAY-n January 2011, page 27). The known cosmetic compositions comprising polyurethanes, in particular, for the cosmetic treatment of keratinous fibers, such as human hairs, still suffer from certain disadvantages. In particular, holding power on keratinous fibers, in particular, on human hair, like Chinese hair especially under high humidity is often too low. Also curl retention at higher humidity, in particular again on Chinese hair is often too low. Moreover, there is a permanent need to reduce flake formation, to make the hair easier to comb, to improve soft feel of the hair, and to improve flexibility of the hair. The object underlying the present invention was in particular to provide a cosmetic composition, comprising a polyurethane polymer that provides strong hold on keratinous fibers, in particular, on human hair, especially on Chinese hair, even under high humidity conditions, without the occurrence of substantial flake formation during combing, thereby improving in particular the curl retention under high humidity. The present inventors surprisingly found that these objects can be solved by providing a cosmetic composition comprising a specific, water dispersible polyurethane. The inventive cosmetic composition provides high holding power and durability on keratinous fibers, in particular, on Chinese hairs in particular under high humidity conditions. In addition flake formation during combing is reduced.

### DETAILED DESCRIPTION OF THE INVENTION

The present invention in one aspect thus relates to a cosmetic composition, comprising at least one water-dispersible polyurethane polymer obtainable by the reaction of at least one isocyanate-functional polyurethane prepolymer A) with at least one isocyanate-reactive component B), wherein

the at least one isocyanate-functional polyurethane prepolymer A) is obtainable by the reaction of at least one polyol component A1), and
at least one polyisocyanate component A2) comprising 60 weight-%, based on the total weight of polyisocyanates in the prepolymer formation reaction, of at least one saturated cycloaliphatic polyisocyanate, and
wherein the molar ratio of the isocyanate groups to the isocyanate-reactive groups in the prepolymer A) formation reaction is at least 1.90, preferably at least 2.00, more preferably at least 2.30 and still more preferably at least 2.50. Also the upper limit of the molar ratio of the isocyanate groups to the isocyanate-reactive groups in the prepolymer A) formation reaction is preferably 4.00, more preferably 3.50. According preferred ranges include in particular 2.00 to 4.00, more preferred 2.30 to 3.50, still more preferred 2.50 to 3.40.

If the molar ratio of the isocyanate groups to isocyanate-reactive groups in the prepolymer A) formation reaction is adjusted accordingly the cosmetic compositions according to the invention provide, in particular, a strong hold on keratinous fibers, in particular, on human hair, especially on Chinese hair, without the occurrence of substantial flake formation during combing, and thereby improve in particular also the curl retention under high humidity conditions.

Said molar ratio of the isocyanate groups to the isocyanate-reactive groups, which are preferably exclusively hydroxyl groups, is suitably calculated from the molecular weights and the functionality of the components A1) and A2) as derived from their structures. Alternatively, they might be also derived from measuring the NCO contents volumetrically in accordance with DIN-EN ISO 11909 of A1, and/or from measuring the OH-number in particular of the polymeric polyols A1) according to DIN 53240.

The polyurethanes used within the context of the present invention are polymeric compounds which have at least two, preferably at least three, repeat units containing urethane groups:

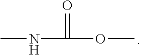

According to the invention, also included are those polyurethanes which, as a result of the preparation, also have repeat units containing urea groups:

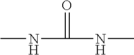

as are formed in particular in the reaction of the isocyanate-terminated prepolymers A) with the amino-functional compounds B).

The polyurethane polymers which are used in the cosmetic compositions according to the invention are water-dispersible. The term “water-dispersible” in the context of the present invention means that they produce a sedimentation-stable dispersion in water, in particular deionized water at 23° C. and no visible phase separation or precipitation takes place for 3 month, the solids content of the dispersion being between 10 and 70 wt % polyurethane polymer. In other words, the polyurethane polymers do not settle out when they are dispersed in water at 23° C. within 3 month. The polyurethane polymer would be considered as non-water dispersible, if of 100 g of polyurethane polymer less than 10 wt % are contained in the aqueous phase after dispersing the polymer in water.

In order to be water-dispersible the polyurethane polymers which are used in the cosmetic compositions normally require the presence of at least one hydrophilizing group, as described below. The at least one hydrophilizing group, is preferably selected from the group consisting of ionic groups, ionogenic (ion-forming) groups and non-ionic or non-ionogenic hydrophilizing groups, which are each as described below. Preferably the at least one hydrophilizing group consists solely of ionic groups and ionogenic groups. The water-dispersible polyurethane polymer comprises the at least one hydrophilizing group suitably in an amount to render the polyurethane polymer water-dispersible. In a preferred embodiment of the invention the at least one hydrophilizing group is introduced into the water-dispersible polyurethane polymer with the isocyanate-reactive component B), which preferably contains at least one hydrophilizing group.

In a preferred embodiment of the cosmetic composition according to the invention the at least one polyisocyanate component A2) comprises ≥60 weight-%, preferably ≥80 weight-%, more preferably 95 to 100 weight-%, based on the total weight of polyisocyanates in the prepolymer formation reaction, of the of at least one saturated cycloaliphatic polyisocyanate.

In a still more preferred embodiment of the cosmetic composition according to the invention the at least one polyisocyanate component A2) is dicyclohexylmethane diisocyanate, in particular 4,4′-diisocyanatodicyclohexylmethane (H12MDI, available for example as Desmodur W®).

In some contrast to the water-dispersible polyurethane polymers, the isocyanate-functional prepolymer A) is preferably a water-insoluble, non-water-dispersible polyurethane prepolymer, which means in particular that the solubility in water of the prepolymer used according to the invention at 23° C. is less than 10 g/litre, more preferably less than 5 g/litre, and that the prepolymer does not produce a sedimentation-stable dispersion in water, in particular deionized water, at 23°. In other words, the prepolymer preferably forms a visible sediment or two separate phases within 24 h at 23° C. upon any attempt to disperse it in water.

Preferably, the polyurethane prepolymer A) used according to the invention has terminal isocyanate groups, i.e. the isocyanate groups are at the chain ends of the prepolymer. All of the chain ends of a prepolymer particularly preferably have isocyanate groups. Furthermore, the polyurethane prepolymer A) used according to the invention preferably has essentially neither ionic nor ionogenic groups, i.e. the content of ionic and ionogenic groups is expediently below 15 milliequivalents per 100 g of polyurethane prepolymer A), preferably below 5 milliequivalents, particularly preferably below 1 milliequivalent and very particularly preferably below 0.1 milliequivalent per 100 g of polyurethane prepolymer A). The term “ionogenic groups” in the context of the present invention shall mean that these groups are potentially capable of forming ionic groups e.g. upon contact with water or upon reaction with acids or bases.

That is, in a preferred embodiment of the invention, the water-dispersible polyurethane polymer is formed by the reaction of a non-water-dispersible isocyanate-functional polyurethane prepolymer A) with at least one isocyanate-reactive component B), which comprises at least one hydrophilizing group, selected from the group consisting of ionic groups, ionogenic groups and non-ionic or non-ionogenic hydrophilizing groups, preferably consisting of ionic groups and ionogenic groups.

In the present invention the polyol component A1) is preferably selected from the group of polymeric polyols, which are intended to mean that they have at least two, more preferably at least three, repeat units joined together. Such polymeric polyols include for example polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols or mixtures thereof. Preferred polymeric polyols include polyether polyols, polycarbonate polyols, polyether-polycarbonate polyols and/or polyester polyols. Among these polyol components A1) the polyester polyols are particularly preferred. The polymeric polyols A1), preferably have number-average molecular weights of from 400 to 8000 g/mol (here and in the case of any molecular weight data below, the number-average molecular weights are determined by gel permeation chromatography relative to polystyrene standard in tetrahydrofuran at 23° C., more specifically according to DIN 55672-1: “gel permeation chromatography, part 1—tetrahydrofuran as eluent (SECurity GPC-System of PSS Polymer Service, flow rate 1.0 ml/min; columns: 2×PSS SDV linear M, 8×300 mm, 5 μm; RID-Detector). Polystyrene samples of known molecular mass are used for calibration. The calculation of the number-average molecular weight is carried out software-supported. Base line points and evaluation limits are determined in accordance with DIN 55672-1, part 1), more preferably 400 to 6000 g/mol and particularly preferably from 600 to 3000 g/mol, and OH functionalities of preferably 1.5 to 6, more preferably 1.8 to 3, particularly preferably from 1.9 to 2.1. Preferred polyester polyols A1) introduce non-hydrophilic soft segments to the polyurethane prepolymer A). The polyester polyol is preferably the sole polyol of the polyol component A1). Preferably the polyester polyols are obtained from aliphatic dicarboxylic acids and aliphatic linear and/or branched diols. Examples for aliphatic dicarboxylic acids include tetrahydrophthalic acid, hexahydrophthalic acid, cyclohexane dicarboxylic acid, adipic acid, azelaic acid, sebacic acid, glutaric acid, maleic acid, fumaric acid, itaconic acid, malonic acid, suberic acid, 2-methylsuccinic acid, 3,3-diethylglutaric acid and/or 2,2-dimethylsuccinic acid, with adipic acid particularly preferred. The corresponding anhydrides can also be used as acid source. The aliphatic dicarboxylic acids may be also used in the form of one or more of their corresponding diester derivatives, particularly their dimethanol or diethanol ester derivatives. Examples for linear aliphatic diols include 1,2-ethanediol (i.e., ethylene glycol), 1,3-propanediol, 1,4-butanediol, 1,5-pentanediol, 1,6-hexanediol, 1,7-heptanediol, 1,8-octanediol, 1,9-nonanediol, 1,10-decanediol and 1,3-butanediol. Preferably, linear aliphatic diols are selected from the group consisting of 1,3- and 1,4-butanediol, 1,6-hexanediol and/or 1,8-octanediol, with 1,6-hexanediol particularly preferred. Examples for branched aliphatic diols include neopentyl glycol, 1,2-propanediol, 2-methyl-1,2-propanediol, 2-methyl-1,3-propanediol, 1,2-butanediol, meso-2,3-butanediol, 2,3-dimethyl-2,3-butanediol (pinacol), 1,2-pentanediol, 2,3-pentanediol, 2,4-pentanediol, 1,4-pentanediol, 2,2,4-trimethyl-1,3-pentanediol, 1,3-hexanediol, 1,4-hexandiol, 1,5-hexanediol, 2,5-hexanediol, 2,5-dimethyl-2,5-hexanediol, 1,3-cyclobutanediol, 2,2,4,4-tetramethylcyclobutanediol, 1,2-cyclopentanediol, 1,3-cyclopentanediol, 1,3-dimethyl-1,3-cyclopentanediol, 1,2-cyclohexanediol, 1,3-cyclohexanediol, 1,4-cyclohexanediol and 1,4-dimethylolcyclohexane. Preferably, the branched aliphatic diols are selected from the group consisting of neopentyl glycol, 2-methyl-1,3-propanediol and/or 2,2,4-trimethyl-1,3-pentanediol, with neopentyl glycol particularly preferred. Preferred polyester polyols as polyol component A1) are obtainable by reacting a mixture comprising at least one aliphatic dicarboxylic acid, and at least one linear and/or branched aliphatic diol. Even more preferred are polyester polyols, obtainable by reacting a mixture comprising at least one aliphatic dicarboxylic acid, at least one linear aliphatic diol, and at least one branched aliphatic diol (i.e. a mixture of a linear and a branched aliphatic diol), and a particularly preferred polyol component A1) is selected from polyester polyols, which are obtainable by reacting a mixture comprising adipic acid, hexanediol and neopentyl glycol. The preferred polyester polyols A1) according to the invention most preferably have a number-average molecular weight of ≥1000 g/mol to ≤2000 g/mol.

In addition to the polymeric polyols it is also possible to use non-polymeric polyols in the preparation of the polyurethane prepolymer A). They have a preferred molecular weight range from 62 to 399 mol/g with up to 20 carbon atoms, such as ethylene glycol, diethylene glycol, triethylene glycol, 1,2-propanediol, 1,3-propanediol, 1,4-butanediol, 1,3-butylene-glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A (2,2-bis(4-hydroxycyclohexyl)propane), trimethylolpropane, trimethylolethane, glycerol, pentaerythritol, and mixtures thereof. Also suitable are ester diols of the specified molecular weight range, such as α-hydroxybutyl ε-hydroxycaproic acid ester, ω-hydroxyhexyl γ-hydroxybutyric acid ester, adipic acid (ß-hydroxyethyl) ester or terephthalic acid bis(ß-hydroxyethyl) ester. In addition, it is also possible to use as non-polymeric polyols monofunctional isocyanate-reactive hydroxyl-group-containing compounds in the preparation of the polyurethane prepolymer A). Examples of such monofunctional compounds are ethanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol, or mixtures thereof.

In a preferred embodiment of the invention, in the manufacture of the polyurethane prepolymer A) by the reaction of the at least one polyol component A1), and at least one polyisocyanate component A2) less than about 10% by weight of such non-polymeric polyols, preferably less than 5% by weight of non-polymeric polyols, in each case based on the total mass of the polyurethane prepolymer A) are used, yet more preferably such non-polymeric polyols are not used for the preparation of the polyurethane prepolymer A).

Further optionally, nonionically hydrophilizing compounds can be used in addition to the polyol component A1), for example, polyoxyalkylene ethers which have isocyanate-reactive groups, such as hydroxy, amino or thiol groups. Preference is given to monohydroxy-functional polyalkylene oxide polyether alcohols having, on statistical average, 5 to 70, preferably 7 to 55, ethylene oxide units per molecule, as are accessible in a manner known per se by alkoxylation of suitable starter molecules (e.g. in Ullmanns Encyclopädie der technischen Chemie [Ullmanns encyclopaedia of industrial chemistry], 4th edition, Volume 19, Verlag Chemie, Weinheim pp. 31-38). Those nonionically hydrophilizing compounds should be used, however, preferably in an amount only that still renders the isocyanate-functional prepolymer A) water-insoluble, non-water-dispersible, as described above. In a preferred embodiment such optional nonionically hydrophilizing compounds are not used.

In a preferred embodiment of the present invention the at least one saturated cycloaliphatic polyisocyanate is used in an amount of preferably ≥70 weight-%, more preferably ≥80 weight-%, more preferably ≥90 weight-%, based on the total weight of polyisocyanates in the prepolymer formation reaction, of the total of the polyisocyanate components A2). Most preferably the polyisocyanate components A2) consist of saturated cycloaliphatic polyisocyanates. Such saturated cycloaliphatic polyisocyanates preferably have at least one, more preferably at least two isocyanate groups attached to a saturated cycloaliphatic ring. They are preferably polyisocyanates or polyisocyanate mixtures of the type specified above preferably with exclusively cycloaliphatically bonded isocyanate groups or mixtures of these and preferably have an average NCO functionality of the mixture of from 2 to 4, preferably 2 to 2.6 and particularly preferably 2 to 2.4, very particularly preferably 2. They include preferably isophorone diisocyanate (IPDI), 1,4-cyclohexylene diisocyanate, 1,3-diisocyanato-2-methylcyclohexane, 1,3-diisocyanato-4-methylcyclohexane, and 4,4′-diisocyanatodicyclohexylmethane (H12MDI), and mixtures thereof, among which dicyclohexylmethane diisocyanate, in particular, 4,4′-diisocyanatodicyclohexylmethane (H12MDI—available e.g. as Desmodur W®) is most preferred. On the basis of these saturated cycloaliphatic polyisocyanates it is also possible to use modified polyisocyanates thereof having uretdione, isocyanurate, urethane, allophanate, biuret, imino-oxadiazinedione and/or oxadiazinetrione structural elements. If employed, additional polyisocyanates may be the aromatic, araliphatic, aliphatic or cycloaliphatic polyisocyanates having an NCO functionality of 2 which are known per se to the person skilled in the art. Examples of such polyisocyanates that can be used in addition to the at least one isocyanate-reactive component A2) include for example 1,4-butylene diisocyanate, 1,6-hexamethylene diisocyanate (HDI), isophorone diisocyanate (IPDI), 2,2,4- and/or 2,4,4-trimethylhexamethylene diisocyanate, 1,4-cyclohexylene diisocyanate, 1,4-phenylene diisocyanate, 2,4- and/or 2,6-tolylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI), and alkyl 2,6-diisocyanatohexanoates (lysine diisocyanates) containing C1-C8-alkyl groups.

In a preferred embodiment of the invention the at least one polyisocyanate component A2) comprises ≥60 weight-%, preferably ≥80 weight-%, more preferably 95 to 100 weight-%, based on the total weight of polyisocyanates in the prepolymer formation reaction, of dicyclohexylmethane diisocyanate (H12MDI):

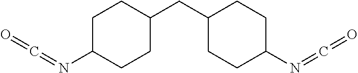 [SMILES: O=C=NC1CCC(CC2CCC(N=C=O)CC2)CC1]

In a most preferred embodiment of the invention the at least one polyisocyanate component A2) is exclusively formed (consisting of) dicyclohexylmethane diisocyanate (H12MDI). As is well-known in the art, H12MDI is a mixture of the three possible conformational isomers in respect to the isocyanate groups) and all isomers and isomer mixtures of H12MDI are within the scope of the present invention. The use of dicyclohexylmethane diisocyanate in the cosmetic compositions of the present invention further improves the hair styling performance of the cosmetic compositions according to the invention. Especially, these cosmetic compositions exhibit higher curl retention especially under high humidity conditions, in particular on Chinese hair, specifically also in comparison to cosmetic compositions that use a polyurethane polymer based on a polyurethane prepolymer A) based on isophorone diisocyanate (IPDI).

Due to an excess of isocyanate groups to the isocyanate-reactive groups, i.e. preferably to the hydroxyl groups, in the manufacture of the polyurethane prepolymers A), the polyurethane prepolymers A) have remaining isocyanate groups, i.e. are isocyanate-functional polyurethane prepolymers A).

In the manufacture of the polyurethane polymer used in the cosmetic compositions of the invention after the manufacture of the, preferably non-water dispersible, polyurethane prepolymers A) subsequently the polyurethane prepolymers A) having isocyanate groups are reacted with at least one isocyanate-reactive component B), whereby the polyurethane polymer used in the cosmetic compositions of the invention is obtained. Preferably as the at least one isocyanate-reactive component B), one or more amino-functional compounds B), such as primary and/or secondary amines and/or diamines, are used. As explained above by using amino-functional compounds B), polymers with urethane and urea groups are formed. The reaction with the at least one isocyanate-reactive component B), preferably a diamine or two or more diamines, particularly preferably takes place with chain extension. In this connection, monofunctional amines can additionally be added as chain terminators to control the molecular weight of the polyurethane polymer.

As component B), in particular amines can be used which have no hydrophilizing groups, like in particular, ionic or ionogenic groups, (aminofunctional component B1) below)), and it is possible to use amines which have at least one hydrophilizing group, preferably selected from the group consisting of ionic groups, ionogenic groups and non-ionic or non-ionogenic hydrophilizing groups, in particular, ionic or ionogenic groups (aminofunctional component B2) below), such as, in particular, anionically hydrophilizing groups. Preferably, in the step of the reaction of the prepolymer with the at least one isocyanate-reactive component B), a mixture of compound B1) and compound B2) is reacted. By using component B1) it is possible to build up a high molar mass without the viscosity of the previously prepared isocyanate-functional prepolymer increasing to a degree which would be an obstacle to processing. By using the combination of components B1) and B2) it is possible to render the polyurethane polymer water-dispersible and also to achieve an optimum balance between hydrophilicity and chain length and thus good substantivity without “build-up” effects arising. The polyurethanes used according to the invention preferably have anionic groups as the hydrophilizing groups, more preferably sulfonate groups. These anionic groups are introduced into the polyurethanes used according to the invention preferably via the amine compound B2) reacted in the step of the reaction of the isocyanate-functional prepolymer A) with the at least one isocyanate-reactive component B). The polyurethanes used according to the invention may also have nonionic or non-ionogenic hydrophilizing groups. However, preferably exclusively ionic or ionogenic groups, in particular sulfonate groups are present in the polyurethanes used according to the invention for the hydrophilization; these are introduced preferably into the polyurethane via the corresponding diamines as component B2).

As component B1), which do not have ionic or ionogenic groups, for example, organic di- or polyamines, such as, for example, 1,2-ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, 4,4-diaminodicyclohexylmethane, hydrazine hydrate, and/or dimethylethylenediamine, can be used as component B1). Moreover, compounds which, besides a primary amino group, also have secondary amino groups or, besides an amino group (primary or secondary), also have OH groups, can also be used as component B1). Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines, such as N-aminoethylethanolamine, ethanolamine, 3-aminopropanol, neopentanolamine. In addition, monofunctional isocyanate-reactive amine compounds can also be used as component B1), such as, for example, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, and suitable substituted derivatives thereof, amidoamines of diprimary amines and monocarboxylic acids, monoketime of diprimary amines, primary/tertiary amines, such as N,N-dimethylaminopropylamine. As component B1), preference is given to using 1,2-ethylenediamine, bis(4-aminocyclohexyl)methane, 1,4-diaminobutane, isophoronediamine, ethanolamine, diethanolamine and diethylenetriamine. Most preferably component B1) is 1,2-ethylenediamine.

Component B) particularly preferably includes at least one aminofunctional component B2) which has ionic or ionogenic groups. Preferred are anionically hydrophilizing compounds as component B2) which preferably contain a sulfonic acid or sulfonate group, particularly preferably a sodium sulfonate group. Suitable anionically hydrophilizing compounds as component B2) are, in particular, the alkali metal salts of mono- and diaminosulfonic acids. Examples of such anionic hydrophilizing agents are salts of 2-(2-aminoethylamino)ethane-sulfonic acid, ethylenediamine-propyl- or -butylsulfonic acid, 1,2- or 1,3-propylenediamine-R-ethylsulfonic acid or taurine. Furthermore, the salt of cyclohexylaminopropanesulfonic acid (CAPS) from WO-A-01/88006 can be used as anionic hydrophilizing agent. Particularly preferred anionic hydrophilizing agents B2) are those which contain sulfonate groups as ionic groups and two amino groups, such as the salts of 2-(2-aminoethylamino)ethylsulfonic acid and 1,3-propylenediamine-β-ethylsulfonic acid. The polyurethanes used according to the invention particularly preferably comprise at least one sulfonate group. Optionally, the anionic group in component B2) may also be a carboxylate or carboxylic acid group. Component B2) is then preferably selected from diaminocarboxylic acids. However, this embodiment is less preferred since carboxylic-acid-based compounds B2) have to be used in higher concentrations. For the hydrophilization, it is also possible to use mixtures of anionic hydrophilizing agents B2) and nonionic hydrophilizing agents B2 as described above in the manufacture of the polyurethane prepolymer, i.e. for example nonionically hydrophilizing compounds like polyoxyalkylene ethers which have isocyanate-reactive groups, such as hydroxy, amino or thiol groups as described above. Preferably such nonionically hydrophilizing compounds B), like polyoxyalkylene ethers, are not used.

As mentioned above it is a preferred embodiment of the invention to use as the isocyanate-reactive component B) a combination of amino-functional compounds B2), which have hydrophilizing groups, preferably ionic and/or ionogenic groups, and amino-functional compounds B1), which have no hydrophilizing groups, like in particular, ionic and/or ionogenic groups. A specifically preferred embodiment is a combination of the compounds B1) and B2) which comprises 2-(2-aminoethylamino)ethane sulfonic acid and/or salts thereof, preferably as the sole compound having a hydrophilizing group. A still more preferred embodiment of the invention uses a combination of 2-(2-aminoethylamino)ethane sulfonic acid and/or salts thereof and ethylene diamine as the isocyanate-reactive component B). Preferably the component B) consists of a combination of 2-(2-aminoethylamino)ethane sulfonic acid and/or salts thereof and ethylene diamine.

In a preferred embodiment for the preparation of the polyurethane prepolymers the components A1) and A2) are used in the following amounts, the individual amounts always adding up to 100% by weight of the polyurethane prepolymer:

50 to 85% by weight of the polyol component A1), more preferably 55 to 80 by weight of the polyol component A1), and still more preferably 60 to 75% by weight of the polyol component A1), and
15% to 50% by weight of the polyisocyanate component A2), more preferably 20% to 45% by weight of the component A2), and still more preferably 25 to 40% by weight of the component A2).

Furthermore in a preferred embodiment for the preparation of the polyurethane polymer the isocyanate-functional polyurethane prepolymer A) and the isocyanate-reactive component B), are used in the following amounts, the individual amounts always adding up to 100% by weight of the polyurethane polymer:

80 to 99% by weight of the isocyanate-functional polyurethane prepolymer A), preferably 85 to 97% by weight of the isocyanate-functional polyurethane prepolymer A), and still more preferably 90 to 97% by weight of the isocyanate-functional polyurethane prepolymer A), and
1 to 20% by weight of the isocyanate-reactive component B), preferably 3 to 15% by weight of the isocyanate-reactive component B), and still more preferably 3 to 10% by weight of the isocyanate-reactive component B).

Regarding the amounts of the preferred combination of the compounds B1) to B2), particularly preferably they are used in a weight ratio B1) to B2) of 2:10 to 8:2, preferably 3:8 to 7:3, still more preferably 4:7 to 6:4.

The preparation of the polyurethane dispersions can be carried out in one or more stage(s) in homogeneous phase or, in the case of multistage reaction, sometimes in disperse phase. Following complete or partial polyaddition from A1) and A2), a dispersion, emulsification or dissolution step preferably takes place. Afterwards, a further polyaddition or modification with the isocyanate-reactive component B), optionally takes place in the disperse phase.

In this connection, all of the methods known from the prior art, such as, for example, prepolymer mixing process, acetone process or melt dispersion process, can be used. Preference is given to using the acetone process.

In a preferred embodiment the polyol component A1) and the polyisocyanate component A2) for the preparation of an isocyanate-functional polyurethane prepolymer are usually initially introduced in their entirety or in part and optionally diluted with a solvent which is miscible with water but inert towards isocyanate groups, and heated to temperatures in the range from 50 to 140° C. To increase the rate of the isocyanate addition reaction, optionally the catalysts known in polyurethane chemistry can be used. Suitable solvents are the customary aliphatic, keto-functional solvents such as acetone, 2-butanone, which can be added not only at the start of the preparation, but optionally in parts also later on. Preference is given to acetone and 2-butanone, and particular preference is given to acetone. The addition of other solvents without isocyanate-reactive groups is also possible, but not preferred. Preferably the polyol component A1) and the polyisocyanate component A2) are reacted in the absence of a solvent until the actual NCO value has dropped below the theoretical NCO value (monitoring by IR spectroscopy). Regarding, the quantitative ratio of isocyanate groups to isocyanate-reactive groups in the prepolymer formation reaction it is referred to the above explanations.

The finished prepolymer A) is then preferably dissolved in solvent, preferably acetone or 2-butanone, most preferably acetone, and then a solution of the isocyanate-reactive component B), preferably a combination of the compounds B1) and B2) and preferably water is metered in. After the reaction of the isocyanate-functional polyurethane prepolymer A) with the at least one isocyanate-reactive component B) (chain-extension) preferably under stirring time for up to 2 hours, the mixture is preferably dispersed by adding water, and the solvent is removed by distillation in vacuo whereby a storage-stable dispersion is obtained, the solids content of which being adjusted by adding water. The solid content of the resulting aqueous dispersion of the polyurethane polymer is preferably adjusted to a range of 10 to 70 wt-%, more preferably 20 to 65 wt-% and most preferably 30 to 60 wrkeach based on the total weight of the aqueous polyurethane dispersion. Preferably such aqueous polyurethane dispersions are used to prepare the cosmetic compositions according to the invention. The solids contents can be ascertained by heating a weighed sample at 125° C. to constant weight. At constant weight, the solid-body content is calculated by reweighing the sample.

In a possible neutralization step partial or complete conversion of potentially anionic groups to anionic groups, bases such as tertiary amines, e.g. trialkylamines having 1 to 12, preferably 1 to 6, carbon atoms, particularly preferably 2 to 3 carbon atoms in each alkyl radical or very particularly preferably alkali metal bases such as the corresponding hydroxides are used. The use of organic amines is not preferred. Neutralizing agents which can be used are preferably inorganic bases, such as aqueous ammonia solution or sodium hydroxide or potassium hydroxide. Preference is given to sodium hydroxide and potassium hydroxide. The quantitative amount of the bases can be 50 and 125 mol %, preferably between 70 and 100 mol % of the quantitative amount of the acid groups to be neutralized.

The neutralization can also take place at the same time as the dispersion by the dispersion water already comprising the neutralizing agent.

In the reaction of the at least one isocyanate-functional polyurethane prepolymer A) with the at least one isocyanate-reactive component B), i.e. the equivalent ratio of NCO-reactive groups of the compounds used for the chain extension and chain termination to free NCO groups of the prepolymer is generally between 40 and 150%, preferably between 50 and 110%, particularly preferably between 60 and 100%.

The residual content of organic solvents in the aqueous polyurethane dispersions prepared in this way is typically less than 10% by weight, preferably less than 3% by weight, preferably less than 3% by weight, still more preferably less than 1% by weight based on the total dispersion. The pH of the aqueous polyurethane dispersions used according to the invention is typically less than 8.0, preferably less than 7.5 and is particularly preferably between 5.5 and 7.5.

Preferably, the polyurethane dispersions used in accordance with the present invention have less than 5% by weight, particularly preferably less than 0.2% by weight, based on the mass of the dispersions, of unbonded organic amines.

The polyurethanes according to the invention are preferably essentially linear molecules, but may also be branched, which is less preferred. The number-average molecular weight (e.g. as determined by GPC using polystyrene standard as described in more detail above) of the polyurethanes preferably used according to the invention is, for example, from about 1.000 to 200.000, preferably from 5.000 to 150.000. Molecular weights above 200.000 can be disadvantageous under certain circumstances since the cosmetic compositions in applications like hair setting compositions are sometimes difficult to wash out.

In order to achieve a good sedimentation stability, the number-average particle size of the special polyurethane dispersions is preferably less than 750 nm, more preferably less than 500 nm, and particularly preferably less than 250 nm determined by means of laser correlation spectroscopy following dilution with deionized water (instrument: Malvern Zetasizer 1000, Malver Inst. Limited).

A particularly preferred cosmetic composition according to the invention comprises at least one polyurethane polymer obtainable by the reaction of at least one isocyanate-functional polyurethane prepolymer A) with at least one isocyanate-reactive component B), wherein the at least one isocyanate-functional prepolymer A) is obtainable by the reaction of at least one polyester polyol A1), obtainable by reacting a mixture comprising adipic acid, hexanediol and neopentyl glycol, and at least one dicyclohexylmethane diisocyanate A2), wherein the molar ratio of the isocyanate groups to the isocyanate-reactive groups in the prepolymer A) formation reaction is at least 2.30 to 3.20, and wherein the at least one isocyanate-reactive component B) comprises 2-(2-aminoethylamino)ethane sulfonic acid and/or salts thereof, and ethylene diamine, which is preferably provided as aqueous dispersion having a solid content of 20 to 65 wt-% based on the total weight of the aqueous polyurethane dispersion.

In a further embodiment the present invention relates the use of the polyurethane polymer as defined above for the manufacture of a cosmetic composition. In a further preferred embodiment of the invention said polyurethane polymer is used as an aqueous dispersion for making the cosmetic composition according to the present invention. Such aqueous dispersions usually have a solid content of generally of 10 to 70 wt-%, more preferably 20 to 65 wt-% and most preferably 30 to 60 wt %. The solids contents are ascertained by heating a weighed sample at 125° C. to constant weight. At constant weight, the solid-body content is calculated by reweighing the sample.

The cosmetic composition according to the invention is preferably suitable for the application on keratinous fibers, the human skin and on nails, more preferably for the application on keratinous fibers and especially on human hair.

Nails in the context of this invention are especially understood to mean human fingernails and/or toenails, but also synthetic nails which have already been secured to the human body or are intended for securing to the human body. Such synthetic nails are based, for example, on materials such as synthetic polymers.

Keratinous fibers especially include human hair and also eyelashes and eyebrows.

Human skin includes also human mucous membranes.

The cosmetic composition according to the invention preferably comprises:
a) one or more cosmetically active ingredients and/or one or more cosmetic auxiliary agents, (“and/or” means that the cosmetic composition can comprise only cosmetically active ingredients, only cosmetic auxiliary agents, or both cosmetically active ingredients and cosmetic auxiliary agents),
b) one or more diluents/solvents,
c) one or more polyurethanes used according to the invention as described before.

The cosmetic compositions according to the invention comprise preferably 0.1 to 40% by weight of the polyurethane described above and in particular 0.5 to 30% by weight, in each case based on the total weight of the cosmetic composition.

Preferably, the cosmetic compositions of the invention, in particular, the cosmetic hair treating formulations comprise the following components:

| | Ingredient | Weight-% |
|---|---|---|
| a) | one or more cosmetically active ingredients, | 0.05 to 65 |
| | and/or one or more cosmetic auxiliary agents | |
| b) | diluents/solvents | 5 to 99.85 |
| c) | polyurethane according to the invention | 0.1 to 30 |

wherein the total weight-percentages add up to 100 weight-% of the cosmetic composition.

The cosmetic composition according to the invention preferably further comprises at least one cosmetically active ingredient and/or one or more auxiliary, preferably cosmetic auxiliary agents that are usually used in cosmetic applications.

Those cosmetically active ingredients and auxiliary agents are preferably selected from the following groups of substances:

film formers, propellant gas, UV absorbers, special-effect constituents, sensory additives, emulsifiers, surfactants, humectants, oils, waxes, thickeners, preservatives, care agents, especially hair care agents, fingernail care agents, plasticizers, coalescing agents, desquamating agents, agents for improving the skin barrier function, depigmenting agents, antioxidants, dermo-decontracting agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting the maturation of the horny envelope, NO-synthase inhibitors, peripheral benzodiazepine receptor (PBR) antagonists, agents for increasing the activity of the sebaceous glands, agents for stimulating the energy metabolism of cells, tensioning agents, lipo-restructuring agents, slimming agents, agents for promoting the cutaneous capillary circulation, calmatives and/or anti-irritants, sebo-regulators or anti-seborrhoeic agents, astringents, cicatrizing agents, anti-inflammatory agents and anti-acne agents, acids, bases, buffers to adjust the pH value, rheology modifiers, structuring agents, fragrances, vitamines, pearlescent agents, gelling agents, trace elements, sequestering agents, antioxidants, anti-hair loss agents, antidandruff agents, ceramides, other styling polymers, fillers, nacres, silicones or silicone derivatives, wetting agents, anticorrosive agents, antiadhesives, combining agents, antistatic agents, lustre agents, proteins and derivatives thereof, amino acids, opacifiers, stabilizers, sequestrants, complexing agents, aesthetic enhancers, fatty acids, fatty alcohols, triglycerides, botanic extracts, clarifying auxiliaries, perfumes, deodorants, anti-transpirants and mixtures thereof, preferably film formers, propellant gas, UV absorbers, special-effect constituents, sensory additives, emulsifiers, surfactants, humectants, oils, waxes, thickeners, preservatives, care agents, especially hair care agents, fingernail care agents, plasticizers and/or coalescing agents.

The most preferred cosmetically active ingredients and auxiliary agents are described in more detail in the following.

The cosmetic compositions of the invention preferably comprise in particular water and optionally a cosmetically suitable diluent or solvent. The preferred solvents are aliphatic alcohols having C2-4 carbon atoms, such as ethanol, isopropanol, t-butanol, n-butanol; polyol, such as propylene glycol, glycerol, ethylene glycol and polyol ethers; acetone;

unbranched or branched hydrocarbons, such as pentane, hexane, isopentane and cyclic hydrocarbons, such as cyclopentane and cyclohexane; and mixtures thereof. A very particularly preferred solvent is ethanol.

Preferably the diluent/solvent, is selected from the group consisting of water, one or more alcohols and mixtures of water and one or alcohols such as the aforementioned aliphatic alcohols.

The cosmetic composition according to the invention preferably comprise solvents/diluents in an amount of 5% by weight to 99.85% by weight, advantageously 10% by weight to 95% by weight, particularly advantageously 30% by weight to 90% by weight, based on the total weight of the composition according to the invention.

Besides the polyurethanes used in accordance with the present invention, the composition according to the invention can comprise further suitable film formers. Further film formers are preferably used in cosmetic compositions for applications on hair, especially in hairstyling compositions.

The concentration of one or more further film formers can be from 0 to 20% by weight and in particular 0.1 to 10% by weight, in each case based on the total weight of the composition. The film former or film formers are advantageously selected from the group of water-soluble or water-dispersible polyurethanes different from the polyurethanes used according to the invention, the polyureas, silicone resins and/or polyesters, and also the nonionic, anionic, amphoteric and/or cationic polymers and their mixtures.

Advantageous nonionic polymers which may be present in compositions according to the invention alone or in a mixture, preferably also with anionic and/or amphoteric and/or zwitterionic polymers, are selected from:
polyalkyloxazolines,
vinyl acetate homopolymers or copolymers. These include, for example, copolymers of vinyl acetate and acrylic acid esters, copolymers of vinyl acetate and ethylene, copolymers of vinyl acetate and maleic acid esters,
acrylic acid ester copolymers, such as, for example, the copolymers of alkyl acrylate and alkyl methacrylate, copolymers of alkyl acrylate and urethanes,
copolymers of acrylonitrile and nonionic monomer selected from butadiene and (meth)acrylate,
styrene homopolymers and copolymers. These include, for example, homopolystyrene, copolymers of styrene and alkyl (meth)acrylate, copolymers of styrene, alkyl methacrylate and alkyl acrylate, copolymers of styrene and butadiene, copolymers of styrene, butadiene and vinylpyridine,
polyamides,
vinyllactam homopolymers or copolymers, such as vinylpyrrolidone homo- or copolymers; these include, for example, polyvinylpyrrolidone, polyvinylcaprolactam, copolymers of N-vinylpyrrolidone and vinyl acetate and/or vinyl propionate in various concentration ratios, polyvinylcaprolactam, polyvinylamides and salts thereof, and also copolymers of vinylpyrrolidone and dimethylaminoethyl methacrylate, terpolymers of vinylcaprolactam, vinylpyrrolidone and dimethylaminoethyl methacrylate,
polysiloxanes,
homopolymers of N-vinylformamide e.g. PVF from AkzoNobel
Polyurethanes, such as Baycusan® C2000 available from Covestro Deutschland AG.

Particularly preferred nonionic polymers are acrylic acid ester copolymers, homopolymers of vinylpyrrolidone and copolymers, polyvinylcaprolactam.

Very particularly preferred nonionic polymers are homopolymers of vinylpyrrolidone, e.g. Luviskol® K from BASF, copolymers of vinylpyrrolidone and vinyl acetate, e.g. Luviskol® VA grades from BASF or PVPVA® S630L from Ashland, terpolymers of vinylpyrrolidone, vinyl acetate and propionate, such as, for example, Luviskol® VAP from BASF and polyvinylcaprolactams, e.g. Luviskol® PLUS from BASF.

Advantageous anionic polymers are homopolymers or copolymers with monomer units containing acid groups which are optionally copolymerized with comonomers which contain no acid groups. Suitable monomers are unsaturated, free-radically polymerizable compounds which have at least one acid group, in particular carboxylic acid, sulfonic acid or phosphonic acid.

Advantageous anionic polymers comprising carboxylic acid group are:
Acrylic acid or methacrylic acid homopolymer or copolymer or the salts thereof. These include, for example, the copolymers of acrylic acid and acrylamides and/or sodium salts thereof, copolymers of acrylic acid and/or methacrylic acid and an unsaturated monomer selected from ethylenes, styrene, vinyl esters, acrylic acid esters, methacrylic acid esters, optionally ethoxylated compounds, copolymers of vinylpyrrolidones, acrylic acid and C1-C20 alkyl methacrylates, e.g. Acrylidone® LM from Ashland, copolymers of methacrylic acid, ethyl acrylates and tert-butyl acrylates, e.g. Luvimer® 100 P from BASF.
Crotonic acid derivative homopolymer or copolymer or the salts thereof. These include, for example, vinyl acetate/crotonic acid, vinyl acetate/acrylate and/or vinyl acetate/vinyl neodecanoate/crotonic acid copolymers, sodium acrylate/vinyl alcohol copolymers,
Unsaturated C4-C8 carboxylic acid derivatives or carboxylic acid anhydride copolymer selected from copolymers of maleic acid or maleic anhydride or fumaric acid or fumaric anhydride or itaconic acid or itaconic anhydride and at least one monomer selected from vinyl esters, vinyl ethers, vinyl halogen derivatives, phenyl vinyl derivatives, acrylic acid, acrylic acid esters or copolymers of maleic acid or maleic anhydride or fumaric acid or fumaric anhydride or itaconic acid or itaconic anhydride and at least one monomer selected from allyl esters, methallyl esters and optionally acrylamides, methacrylamides, alpha-olefin, acrylic acid esters, methacrylic acid esters, vinylpyrrolidones. Further preferred polymers are methyl vinyl ether/maleic acid copolymers, which are formed by hydrolysis of vinyl ether/maleic anhydride copolymers. These polymers can also be partially esterified (ethyl, isopropyl or butyl esters) or partially amidated.
Water-soluble or -dispersible anionic polyurethanes, e.g. Luviset® PUR from BASF and Dynamx® from AkzoNobel, Baycusan® C1000, Baycusan® C1001, Baycusan® C1003, Baycusan® C1004, Baycusan® C1008, from Covestro Deutschland AG, which are different from the polyurethanes according to the invention,
   where this list is of course not intended to be limiting.

Advantageous anionic polymers containing sulfonic acid group are salts of polyvinylsulfonic acid, salts of polystyrene sulfonic acid, such as, for example, sodium polystyrene sulfonate or salts of polyacrylamide sulfonic acid.

Particularly advantageous anionic polymers are acrylic acid copolymers, crotonic acid derivative copolymer, copolymers of maleic acid or maleic anhydride or fumaric acid or fumaric anhydride or itaconic acid or itaconic anhydride and at least one monomer selected from vinyl esters, vinyl ethers, vinyl halogen derivatives, phenyl vinyl derivatives, acrylic acid, acrylic acid esters and salts of polystyrene sulfonic acid.

Very particularly advantageous anionic polymers are acrylate copolymers, e.g. Luvimer from BASF, ethyl acrylate/N-tert-butylacrylamide/acrylic acid copolymers ULTRAHOLD® STRONG from BASF, VA/crotonate/vinyl neodecanoate copolymer, e.g. Resyn 28-2930 from AkzoNobel, copolymers such as, for example, copolymers of methyl vinyl ether and maleic anhydride partially esterified e.g. GANTREZ® from Ashland and sodium polystyrene sulfonates e.g. Flexan 130 from AkzoNobel.

Advantageous amphoteric polymers can be selected from the polymers which contain units A and B distributed randomly in the polymer chain, where A means a unit which is derived from a monomer with at least one basic nitrogen atom, and B is a unit which originates from an acidic monomer which has one or more carboxy groups or sulfonic acid groups, or A and B can be groups which are derived from zwitterionic carboxybetaine monomers or sulfobetaine monomers; A and B can also be a cationic polymer chain which contains primary, secondary, tertiary or quaternary groups, in which at least one amino group carries a carboxy group or sulfonic acid group which is bonded via a hydrocarbon group, or B and C are part of a polymer chain with ethylene-α,β-dicarboxylic acid unit in which the carboxylic acid groups have been reacted with a polyamine which contains one or more primary or secondary amino groups.

Particularly advantageous amphoteric polymers are:
Polymers which are formed during the copolymerization of a monomer derived from a vinyl compound with carboxy group, such as, in particular, acrylic acid, methacrylic acid, maleic acid, α-chloroacrylic acid, and a basic monomer which is derived from a vinyl compound which is substituted and contains at least one basic atom, such as, in particular, dialkylaminoalkyl methacrylate and acrylate, dialkylaminoalkylmethacrylamide and -acrylamide. Such compounds have been described in the U.S. Pat. No. 3,836,537.
Polymers with units which are derived from: a) at least one monomer which is selected from the acrylamides or methacrylamides which are substituted on the nitrogen atom by an alkyl group, b) at least one acidic comonomer which contains one or more reactive carboxy groups, and c) at least one basic comonomer, such as esters of acrylic acid and methacrylic acid with primary, secondary, tertiary and quaternary amino substituents and the quartenization product of dimethylaminoethyl methacrylate with dimethyl sulphate or diethyl sulphate.

N-substituted acrylamides or methacrylamides particularly preferred according to the invention are compounds whose alkyl groups contain 2 to 12 carbon atoms, particularly N-ethylacrylamide, N-t-butylacrylamide, N-t-octylacrylamide, N-octylacrylamide, N-decylacrylamide, N-dodecylacrylamide, and the corresponding methacrylamides.

The acidic comonomers are selected in particular from acrylic acid, methacrylic acid, crotonic acid, itaconic acid, maleic acid, fumaric acid and the alkyl monoesters having 1 to 4 carbon atoms of maleic acid, maleic anhydride, fumaric acid or fumaric anhydride.

Preferred basic comonomers are aminoethyl methacrylate, butylaminoethyl methacrylate, N,N-dimethylaminoethyl methacrylate, N-t-butylaminoethyl methacrylate.
Crosslinked and completely or partially acylated polyamino amides which are derived from polyamino amides of the following general formula:

—[CO—R—CO—Z]—

in which R is a divalent group which is derived from a saturated dicarboxylic acid, an aliphatic mono- or dicarboxylic acid with ethylenic double bond, an ester of these acids with a lower alkanol having 1 to 6 carbon atoms or a group which is formed upon the addition of one of these acids onto a bis-primary or bis-secondary amine, and Z is a group which is derived from a bis-primary, mono- or bis-secondary polyalkylenepolyamine, and preferably: a) in quantitative fractions from 60 to 100 mol % the groups —NH—[(CH₂)ₓ—NH—]ₚ— where x=2 and p=2 or 3 or x=3 and p=2, where this group is derived from diethylenetriamine, triethylenetetramine or dipropylenetriamine; b) in quantitative fractions of from 0 to 40 mol % the group —NH—[(CH₂)ₓ—NH—]ₚ—, in which x=2 and p=1, which is derived from ethylenediamine, or the group which originates from piperazine:

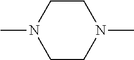

c) in quantitative fractions of from 0 to 20 mol %, the group —H—(CH₂)₆—NH—, which is derived from hexamethylenediamine, where these polyaminoamides are crosslinked by adding a bifunctional crosslinking agent, which is selected from epihalohydrins, diepoxides, dianhydrides and bis-unsaturated derivatives, in an amount of from 0.025 to 0.35 mol of crosslinking agent per amino group of the polyaminoamide, and acylated with acrylic acid, chloroacetic acid or an alkanesultone or salts thereof.

The saturated carboxylic acids are preferably selected from the acids having 6 to 10 carbon atoms, such as adipic acid, 2,2,4-trimethyladipic acid and 2,4,4,-trimethyladipic acid, terephthalic acid; acids with ethylenic double bond, such as, for example, acrylic acid, methacrylic acid and itaconic acid.

The alkanesultones used in the acylation are preferably propanesultone or butanesultone, the salts of the acylating agents are preferably the sodium salts or potassium salts.
Polymers with zwitterionic units of the following formula:

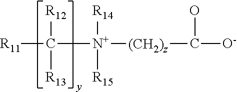

in which R₁₁ is a polymerizable unsaturated group, such as acrylate, methacrylate, acrylamide or methacrylamide, y and z are integers from 1 to 3, R₁₂ and R₁₃ are a hydrogen atom, methyl, ethyl or propyl, R₁₄ and R₁₅ are a hydrogen atom or an alkyl group which is selected such that the sum of the carbon atoms R₁₄ and R₁₅ does not exceed 10.

Polymers which contain such units can also have units which originate from non-zwitterionic monomers, such as dimethyl- and diethylaminoethyl acrylate or dimethyl- and diethylaminoethyl methacrylate or alkyl acrylates or alkyl methacrylates, acrylamides or methacrylamides or vinyl acetate.
Polymers which are derived from chitosan and contain monomer units which correspond to the following formulae:

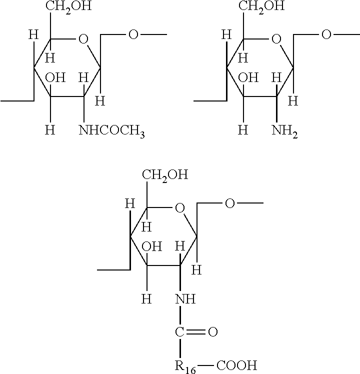

where the first unit is present in quantitative fractions of from 0 to 30%, the second unit is present in quantitative fractions of from 5 to 50% and the third unit is present in quantitative fractions of from 30 to 90%, with the proviso that, in the third unit, R₁₆ is a group of the following formula:

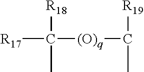

in which: if q=0, the groups R₁₇, R₁₈ and R₁₉, which are identical or different, are in each case a hydrogen atom, methyl, hydroxy, acetoxy or amino, a monoalkylamine radical or a dialkylamine radical which is optionally interrupted by one or more nitrogen atoms and/or optionally one or more of the groups amino, hydroxy, carboxy, alkylthio, sulfonic acid, alkylthio, whose alkyl group carries an amino radical, where at least one of the groups R₁₇, R₁₈ and R₁₉ is in this case a hydrogen atom; or if q=1, the groups R₁₇, R₁₈ and R₁₉ are in each case a hydrogen atom, and also the salts which form these compounds with bases or acids.
Polymers which correspond to the following general formula and which are described, for example, in the French patent 1 400 366:

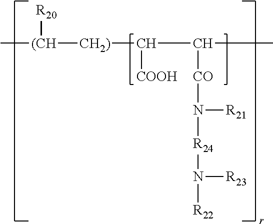

in which R₂₀ is a hydrogen atom, CH₃O, CH₃CH₂O or phenyl, R₂₁ is a hydrogen atom or a lower alkyl group, such as methyl or ethyl, R₂₂ is a hydrogen atom or a lower C₁₋₆-alkyl group, such as methyl or ethyl, R₂₃ is a lower C₁₋₆-alkyl group, such as methyl or ethyl or a group of the formula: —R₂₄—N(R₂₂)₂, where R₂₄ is a group —CH₂—CH₂, —CH₂—CH₂—CH₂— or —CH₂—CH(CH₃)— and where R₂₂ has the meanings given above.
Polymers which can be formed during the N-carboxyalkylation of chitosan, such as N-carboxymethyl chitosan or N-carboxybutyl chitosan.
Amphoteric polymers of the type -D-X-D-X, which are selected from:
a) polymers which are formed through the action of chloroacetic acid or sodium chloroacetate on compounds with at least one unit of the following formula: D-X-D-X, in which D is the group

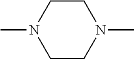

and X is the symbols E or E′, where E or E′, which are identical or different, are a divalent group, which is a straight-chain or branched alkylene group having up to 7 carbon atoms in the main chain, which is present in unsubstituted form or is substituted by hydroxy groups and can contain one or more oxygen atoms, nitrogen atoms or sulphur atoms and 1 to 3 aromatic and/or heterocyclic rings; where the oxygen atoms, nitrogen atoms and sulphur atoms are present in the form of the following groups: ether, thioether, sulfoxide, sulfone, sulfonium, alkylamine, alkenylamine, hydroxy, benzylamine, amine oxide, quaternary ammonium, amide, imide, alcohol, ester and/or urethane.
b) Polymers of the formula D-X-D-X, in which D is the group

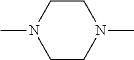

and X is the symbol E or E′ and at least once E′; where E has the meanings given above and E′ is a divalent group, which is a straight-chain or branched alkylene group having up to 7 carbon atoms in the main chain, which is present in unsubstituted form or is substituted by one or more hydroxy groups and contains one or more nitrogen atoms, where the nitrogen atom is substituted by an alkyl group, which is optionally interrupted by an oxygen atom and obligatorily contains one or more carboxy functions or one or more hydroxy functions and is betainized through reaction with chloroacetic acid or sodium chloroacetate.
Alkyl(C₁₋₅) vinyl ether/maleic anhydride copolymers which are partially modified by semiamidation with an N,N-dialkylaminoalkylamine, such as N,N-dimethylaminopropylamine or an N,N-dialkylaminoalcohol. These polymers can also contain further comonomers, such as vinylcaprolactam.

Very particularly advantageous amphoteric polymers are, for example, the copolymers octylacrylamide/acrylates/butylaminoethyl methacrylate copolymers which are commercially available under the names AMPHOMER®, AMPHOMER® LV 71 or BALANCE® 47 from AkzoNobel, and methyl methacrylate/methyl dimethylcarboxymethylammonium ethyl methacrylate copolymers.

It is optionally advantageous to neutralize the anionic and amphoteric polymers using suitable bases in order to improve their solubility and/or dispersibility in water.

The following bases can be used as neutralizing agents for polymers which contain acid groups: hydroxides whose cation is an ammonium or an alkali metal, such as, for example, NaOH or KOH.

Other neutralizing agents are primary, secondary or tertiary amines, amino alcohols or ammonia. Preference is given here to 2-amino-2-methyl-1,3-propanediol (AMPD), 2-amino-2-ethyl-1,3-propanediol (AEPD), 2-amino-2-methyl-1-propanol (AMP), 2-amino-1-butanol (AB), 2-amino-1,3-propanediol, monoethanolamine (MEA), diethanolamine (DEA), triethanolamine (TEA), monoisopropanolamine (MIPA), diisopropanolamine (DIPA), triisopropanolamine (TIPA), dimethyllaurylamine (DML), dimethylmyristalamine (DMM) and dimethylstearamine (DMS).

The neutralization can be partial or complete depending on the intended application.

In some cases, it is also possible, but less preferred, to use cationic polymers, such as, for example, polymers which contain primary, secondary, tertiary and/or quaternary amino groups which are bonded as part of the polymer chain or directly to the polymer chain.

As a further component of the cosmetic composition a propellant gas can be used for example in an amount of from 0 to 70% by weight and particularly preferably in a concentration of from 0.1 to 50% by weight, based on the total weight of the formulation. The propellant gases preferred according to the invention are dimethyl ether and 1,2-difluoroethane (propellant HFC152 A).

However, hydrocarbons such as propane, isobutane and n-butane, and mixtures thereof, compressed air, carbon dioxide, nitrogen, nitrogen dioxide and dimethyl ether, and mixtures of all of these gases can also be used advantageously according to the invention. The person skilled in the art is of course aware that there are propellant gases which are nontoxic per se which would in principle be suitable for realizing the present invention in the form of aerosol preparations, but which nevertheless have to be dispensed with on account of an unacceptable impact on the environment or other accompanying circumstances, in particular fluorocarbons and chlorofluorocarbons (CFCs), such as, for example, 1,2-difluoroethane (propellant 152 A).

The cosmetic composition according to the invention can optionally also comprise UV absorbers, where the total amount of the sunscreen filter is 0% by weight to 35% by weight, 0.1% by weight to 25% by weight, based on the total weight of the composition according to the invention. The UV Absorbers (or UV filters) can in particular be selected from the organic filters, the physical filters and mixtures thereof.

UV absorbers are preferably used in cosmetic compositions that are sun care products, but can also be used in any other cosmetic compositions for the application on skin or hair.

The composition according to the invention can comprise UV-A filters, UV-B filters or broadband filters. The UV filters used can be oil-soluble or water-soluble. The list of specified UV filters below is of course not limiting.

Examples of the UV-B filters are:
(1) salicylic acid derivatives, particularly homomenthyl salicylate, octyl salicylate and 4-isopropylbenzyl salicylate;
(2) cinnamic acid derivatives, in particular 2-ethylhexyl p-methoxycinnamate, which is available from Givaudan under the name Parsol MCX® and isopentyl 4-methoxycinnamate;
(3) liquid β,β′-diphenylacrylate derivatives, in particular 2-ethylhexyl α,β′-diphenylacrylate or octocrylene, which is available from BASF under the name UVINUL N539®;
(4) p-aminobenzoic acid derivatives, in particular 2-ethylhexyl 4-(dimethylamino)benzoate, amyl 4-(dimethylamino)benzoate;
(5) 3-benzylidenecamphor derivatives, in particular 3-(4-methylbenzylidene)camphor which is commercially available from Merck under the name EUSOLEX 6300®, 3-benzylidenecamphor, benzylidenecamphor sulphonic acid and polyacrylamidomethylbenzylidenecamphor;
(6) 2-phenylbenzimidazole-5-sulphonic acid, which is available under the name EUSOLEX 232® from Merck;
(7) 1,3,5-triazine derivatives, in particular: 2,4,6-tris[p-(2′-ethylhexyl-1′-oxycarbonyl)anilino]-1,3,5-triazine, which is supplied by BASF under the name UVINUL T150®, and -dioctylbutamidotriazone, which is supplied by Sigma 3V under the name UVASORB HEB®;
(8) esters of benzalmalonic acid, in particular di(2-ethylhexyl) 4-methoxybenzalmalonate and 3-(4-(2,2-bisethoxycarbonylvinyl)-phenoxy)propenyl)methoxysiloxane/dimethylsiloxane copolymer, which is available from Roche Vitamines under the name Parsol® SLX; and
(9) the mixtures of these filters.
Examples of UV-A filters are:
(1) dibenzoylmethane derivatives, particularly 4-(t-butyl)-4′-methoxydibenzoylmethane, which is supplied by Givaudan under the name PARSOL 1789® and 1-phenyl-3-(4′-isopropylphenyl)propane-1,3-dione;
(2) benzene-1,4-[di(3-methylidenecamphor-10-sulphonic acid)], optionally completely or partially neutralized, commercially available under the name MEXORYL SX® from Chimex.
(3) hexyl 2-(4′-diethylamino-2′-hydroxybenzoyl)benzoate (also aminobenzophenone);
(4) silane derivatives or polyorganosiloxanes with benzophenone groups;
(5) anthranilates, particularly menthyl anthranilate, which is supplied by Symrise under the name NEO HELIOPAN MA®;
(6) compounds which contain at least two benzoazolyl groups or at least one benzodiazolyl group per molecule, in particular 1,4-bis-benzimidazolylphenylene-3,3′,5,5′-tetrasulphonic acid and its salts, which are commercially available from Symrise;
(7) silicon derivatives of benzimidazolylbenzazoles, which are N-substituted, or of benzofuranylbenzazoles, in particular: —2-[1-[3-[1,3,3,3-tetramethyl-l-[(trimethylsilyl)oxy]disiloxanyl]propyl]-1H-benzimidazol-2-yl]benzoxazole; —2-[1-[3-[1,3,3,3-tetramethyl-l-[(trimethylsilyl)oxy]disiloxanyl]propyl]-1H-benzimidazol-2-yl]benzothiazole; —2-[1-(3-trimethylsilanylpropyl)-1H-benzimidazol-2-yl]benzoxazole; —6-methoxy-1,1′-bis(3-trimethylsilanylpropyl)1H,1′H-[2,2′]dibenzimidazolylbenzoxazole; —2-[1-(3-trimethylsilanylpropyl)-1H-benzimidazol-2-yl]benzothiazole; which are described in the patent application EP-A-1 028 120;
(8) triazine derivatives, in particular 2,4-bis[5-1(dimethylpropyl)benzoxazol-2-yl-(4-phenyl)imino]-6-(2-ethylhexyl)imino-1,3,5-triazine, which is supplied by 3V under the name Uvasorb® K2A; and
(9) mixtures thereof.

Examples of broadband filters are:
(1) benzophenone derivatives, for example—2,4-dihydroxybenzophenone (benzophenone-1); —2,2′,4,4′-tetrahydroxybenzophenone (benzophenone-2); —2-hydroxy-4-methoxybenzophenone (benzophenone-3), available from BASF under the name UNIVNUL M40®; —2-hydroxy-4-methoxybenzophenone-5-sulphonic acid (benzophenone-4), and its sulphonate form (benzophenone-5), commercially available from BASF under the name UVINUL MS40®; —2,2′-dihydroxy-4,4′-dimethoxybenzophenone (benzophenone-6-); —5-chloro-2-hydroxybenzophenone (benzophenone-7-); —2,2′-dihydroxy-4-methoxybenzophenone (benzophenone-8); —the disodium salt of 2,2′-dihydroxy-4,4′-dimethoxybenzophenone-5,5′-disulphonic acid (benzophenone-9-); —2-hydroxy-4-methoxy-4′-methylbenzophenone (benzophenone-10);
—benzophenone-11; —2-hydroxy-4-(octyloxy)benzophenone (benzophenone-12).
(2) triazine derivatives, in particular 2,4-bis{[4-2-ethylhexyloxy)-2-hydroxy]-phenyl}-6-(4-methoxyphenyl)-1,3,5-triazine, which is supplied by Ciba Geigy under the name TINOSORB S®, and 2,2′-methylenebis[6-(2H-benzotriazol-2-yl)4-(1,1,3,3-tetramethylbutyl)phenol], which is available from Ciba Geigy under the name TINOSORB M®; and
(3) 2-(1H-benzotriazol-2-yl)-4-methyl-6-[2-methyl-3-[1,3,3,3-tetramethyl-1-[(trimethylsilyl)oxy]disiloxanyl]propyl]phenol with the INCI name Drometrizole Trisiloxane.

It is also possible to use a mixture of two or more filters and a mixture of UV-B filters, UV-A filters and broadband filters, and also mixtures with physical filters.

Of the physical filters, the sulphates of barium, oxides of titanium (titanium dioxide, amorphous or crystalline in the form of rutile and/or anatase), of zinc, of iron, of zirconium, of cerium, silicon, manganese or mixtures thereof may be given. The metal oxides can be present in particle form with a size in the micrometre range or nanometre range (nanopigments). The average particle sizes for the nanopigments are, for example, 5 to 100 nm.

The composition according to the invention may comprise a special-effect constituent. The constituents mentioned may especially have a colouring effect or else provide other effects, such as sparkle and/or metallic effects. Those constituents are preferably used in cosmetic compositions which are used as decorative cosmetics on skin and keratinous fibers and also in cosmetic compositions for the application on nails. Preferably, the composition according to the invention comprises at least one dye which is preferably selected from the group of lipophilic dyes, hydrophilic dyes, pigments, paillettes and mother of pearl.

Particularly advantageously in accordance with the invention, the concentration of the special-effect constituents is ≥0% and ≤50% by weight, particularly advantageously ≥0.1% and ≤30% by weight, very particularly advantageously ≥0.5% and ≤15% by weight, based in each case on the total weight of the composition.

For example, it is possible to use lipophilic dyes, such as Sudan I (yellow), Sudan II (orange), Sudan III (red), Sudan IV (scarlet), DC Red 17, DC Green 6, β-carotene, soya oil, DC Yellow 11, DC Violet 2, DC Orange 5 and DC Yellow 10.

For example, it is possible to use hydrophilic dyes, such as beetroot juice and methylene blue.

The pigments may in principle be any inorganic or organic pigments which are used in cosmetic or dermatological compositions. The pigments used in accordance with the invention may, for example, be white or coloured, and they may be encased or coated with a hydrophobic coating composition or be uncoated.

Advantageously, the pigments are selected from the group of the metal oxides, such as the oxides of iron (especially the oxides that are yellow, red, brown or black in colour), titanium dioxide, zinc oxide, cerium oxide, zirconium oxide, chromium oxide; manganese violet, ultramarine blue, Prussian blue, ultramarine and iron blue, bismuth oxide chloride, mother of pearl, mica pigments coated with titanium or bismuth oxide chloride, coloured pearlescent pigments, for example titanium-mica pigments comprising iron oxides, titanium-mica pigments, especially comprising iron blue or chromium oxide, titanium-mica pigments comprising an organic pigment of the aforementioned type, and pearlescent pigments based on bismuth oxide chloride, carbon black, the pigments of the D&C type, such as D&C Red No. 5, 6, 7, 10, 11, 12, 13, 34, D&C Yellow Lake No. 5 and D&C Red Lake No. 2, and the coating materials based on cochineal red, barium, strontium, calcium and aluminium, and mixtures thereof.

The cosmetic composition according to the invention can also comprise sensory additives. Sensory additives are to be understood as meaning colourless or white, mineral or synthetic, lamellar, spherical or elongated inert particles or a nonparticulate sensory additive which, for example, further improve the sensory properties of the formulations and, for example, leave behind a velvety or silky skin feel. Sensory additives are especially used in compositions for the application on skin.

The sensory additives can be present in the composition according to the invention, for example, in an amount of from 0 to 10% by weight, based on the total weight of the composition, and preferably from 0.1 to 7%.

Advantageous particulate sensory additives within the context of the present invention are talc, mica, silicon dioxide, kaolin, starch and derivatives thereof (for example tapioca starch, distarch phosphate, aluminium and sodium starch octenyl succinate and the like), pyrogenic silica, pigments which have neither primarily a UV-filter effect nor colouring effect (such as e.g. boron nitride etc.), boron nitride, calcium carbonate, dicalcium phosphate, magnesium carbonate, magnesium hydrogencarbonate, hydroxyapatites, microcrystalline cellulose, powders of synthetic polymers, such as polyamides (for example the polymers available under the trade name “Nylon®”), polyethylene, poly-β-alanine, polytetrafluoroethylene (“Teflon®”), polyacrylate, polyurethane, lauroyl-lysine, silicone resin (for example the polymers available under the trade name “Tospearl®” from Kobo Products Inc.), hollow particles of polyvinylidene/acrylonitriles (Expancel® from Akzo Nobel) or hollow particles of silicon oxide (Silica Beads® from MAPRECOS).

Advantageous nonparticulate sensory additives can be selected from the group of dimethiconols (e.g. Dow Corning 1503 Fluid from Dow Corning Ltd.), silicone copolymers (e.g. divinyldimethicone/dimethicone copolymer, Dow Corning HMW 2220 from Dow Corning Ltd.) or silicone elasters (e.g. dimethicone crosspolymer, Dow Corning 9040 Silicone Elastomer Blend from Dow Corning Ltd.).

The cosmetic compositions according to the invention can also comprise one or more emulsifiers. The emulsifiers can be chosen according to the desired application. Especially useful are emulsifiers in cosmetic compositions for the application on skin.

The cosmetic composition according to the invention can comprise one or a mixture of different emulsifiers in an amount of 0% by weight to 20% by weight, advantageously 0.1% by weight to 15% by weight, particularly advantageously 1% by weight to 10% by weight, based on the total weight of the composition according to the invention.

Emulsifiers can be selected from among amphoteric, anionic, cationic and nonionic emulsifiers and can be used alone or as a mixture. The emulsifiers are appropriately selected according to the emulsion to be obtained (W/O or O/W). The emulsions may also contain stabilizers of other types, for instance waxes, fillers, gelling polymers or thickeners.

As emulsifying surfactants that may be used for the preparation of the W/O emulsions, examples include sorbitan, glycerol or sugar alkyl esters or ethers; silicone surfactants, for instance dimethicone copolyols, such as the mixture of cyclomethicone and of dimethicone copolyol, marketed under the trademark DC 5225 C by Dow Corning, and alkyldimethicone copolyols such as laurylmethicone copolyol marketed under the trademark Dow Corning 5200 Formulation Aid by Dow Corning; cetyldimethicone copolyol, such as the product marketed under the trademark Abil EM 90R by Evonik, and the mixture of cetyldimethicone copolyol, of polyglyceryl isostearate (4 mol) and of hexyl laurate, marketed under the trademark Abil WE O9 by Evonik. Other examples are the silicone emulsifiers from Momentive under the trademarks SF1528, SF1540, Silform EOF, Silform 60-A.

One or more co-emulsifiers may also be added thereto, which may be selected advantageously from the group comprising polyol alkyl esters.

Polyol alkyl esters that are especially exemplary include polyethylene glycol esters, for instance PEG-30 dipolyhydroxystearate, such as the product marketed under the trademark Arlacel P135 by Croda Glycerol and/or sorbitan esters that are especially exemplary include, for example, polyglyceryl isostearate, such as the product marketed under the trademark Isolan GI 34 by Evonik, sorbitan isostearate, such as the product marketed under the trademark Arlacel 987 by Croda, sorbitan glyceryl isostearate, such as the product marketed under the trademark Arlacel 986 by Croda, and mixtures thereof.

For O/W emulsions, examples of emulsifiers include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters, for instance the mixture PEG-100 stearate/glyceryl stearate marketed, for example, by Croda under the trademark Arlacel 165; oxyalkylenated (oxyethylenated and/or oxypropylenated) fatty alkyl ethers; sugar esters, for instance sucrose stearate; fatty alkyl ethers of sugars, especially polyalkylglucosides (APG) such as decylglucoside and laurylglucoside marketed, for example, by BASF under the respective names Plantaren 2000 and Plantaren 1200, cetostearyl glucoside optionally as a mixture with cetostearyl alcohol, marketed, for example, under the trademark Montanov 68 by SEPPIC, under the trademark Tegocare CG90 by Evonik and under the trademark Emulgade KE3302 by BASF, and also arachidyl glucoside, for example in the form of a mixture of arachidyl alcohol, behenyl alcohol and arachidyl glucoside, marketed under the trademark Montanov 202 by SEPPIC. Examples of silicones emulsifiers, suitable for O/W emulsions are the polyether siloxane copolymers under the trademarks, SF1188A, SF1288, Silsoft 880, Silsoft 860, Silsoft 440, Silsoft 895, Silsoft 900.

Further emulsion stabilizers that will be used more particularly are isophthalic acid or sulfoisophthalic acid polymers, and in particular phthalate/sulfoisophthalate/glycol copolymers, for example the diethylene glycol/phthalate/isophthalate/1,4-cyclohexanedimethanol copolymer (INCI name: Polyester-5) marketed under the trademark Eastman AQ Polymer (AQ35S, AQ38S, AQ55S and AQ48 Ultra) by Eastman Chemical.

Other suitable emulsifiers are the amino-based emulsifiers, such as sodium stearoyl glutamate and phospholipids such as lecithin, hydroxylated lecithin.

The cosmetic compositions according to the invention can also comprise surfactants which are selected from the group of anionic, cationic, nonionic and/or amphoteric surfactants. Surfactants are especially useful in compositions for the application on hair.

The cosmetic composition according to the invention can comprise one or a mixture of different surfactants in an amount of 0% by weight to 20% by weight, advantageously 0.1% by weight to 15% by weight, particularly advantageously 1% by weight to 10% by weight, based on the total weight of the composition according to the invention.

Advantageous anionic surfactants within the context of the present invention are:

acylamino acids and salts thereof; such as acylglutamates, in particular sodium acyl glutamate and sarcosinates, for example myristoyl sarcosine, TEA lauroyl sarcosinate, sodium lauroyl sarcosinate and sodium cocoyl sarcosinate;
sulphonic acids and salts thereof, such as acyl isethionates, for example sodium or ammonium cocoyl isethionate, sulphosuccinates, for example dioctyl sodium sulphosuccinate, disodium laureth sulphosuccinate, disodium lauryl sulphosuccinate and disodium undecylenamido MEA sulphosuccinate, disodium PEG-5 lauryl citrate sulphosuccinate and derivatives;
sulphuric acid esters, such as alkyl ether sulphate, for example sodium, ammonium, magnesium, MIPA, TIPA laureth sulphate, sodium myreth sulphate and sodium C12-13 pareth sulphate, and alkyl sulphates, for example sodium, ammonium and TEA lauryl sulphate;
taurates, for example sodium lauroyl taurate and sodium methylcocoyl taurate or ether carboxylic acids, for example sodium laureth-13 carboxylate and sodium PEG-6 cocamide carboxylate, sodium PEG-7 olive oil carboxylate;
phosphoric acid esters and salts, such as, for example, DEA oleth-10 phosphate and dilaureth-4 phosphate;
alkylsulphonates, for example sodium coconut monoglyceride sulphate, sodium C012 to C14-olefinsulphonate, sodium lauryl sulphoacetate and magnesium PEG-3 cocamidosulphate; acyl glutamates, such as di-TEA palmitoyl aspartate and sodium caprylic/capric glutamate, acyl peptides, for example palmitoyl hydrolysed milk protein, sodium cocoyl hydrolysed soya protein and sodium/potassium cocoyl hydrolysed collagen;
carboxylic acids and derivatives, such as, for example, lauric acid, aluminium stearate, magnesium alkanolate and zinc undecylenate, ester carboxylic acids, for example calcium stearoyllactylate, laureth-6 citrate and sodium PEG-4 lauramide carboxylate; and alkylarylsulphonates.

Within the context of the present invention, advantageous cationic surfactants are quaternary surfactants. Quaternary surfactants contain at least one N atom which is covalently bonded to 4 alkyl or aryl groups. Alkylbetaine, alkylamidopropylbetaine and alkylamidopropylhydroxysultaine, for example, are advantageous.

Further advantageous cationic surfactants within the context of the present invention are also alkylamines, alkylimidazoles and ethoxylated amines and in particular salts thereof.

Advantageous amphoteric surfactants within the context of the present invention are acyl/dialkylethylenediamines, for example sodium acyl amphoacetate, disodium acyl amphodipropionate, disodium alkyl amphodiacetate, sodium acyl amphohydroxypropylsulphonate, disodium acyl amphodiacetate, sodium acyl amphopropionate, and N-coconut fatty acid amidoethyl N-hydroxyethylglycinate sodium salts.

Further advantageous amphoteric surfactants are N-alkylamino acids, for example aminopropylalkylglutamide, alkylaminopropionic acid, sodium alkylimidodipropionate and lauroamphocarboxyglycinate.

Advantageous active nonionic surfactants within the context of the present invention are alkanolamides, such as cocamides MEAIDEA/MIPA, esters which are formed by esterification of carboxylic acids with ethylene oxide, glyceryl, sorbitan or other alcohols, ethers, for example ethoxylated alcohols, ethoxylated lanoline, ethoxylated polysiloxanes, propoxylated POE ethers, alkyl polyglycosides, such as lauryl glucoside, decyl glycoside and cocoglycoside, glycosides with an HLB value of at least 20 (e.g. Belsil® SPG 128V from Wacker).

Further advantageous nonionic surfactants are alcohols and amine oxides, such as cocoamidopropylamine oxide.

Among the alkyl ether sulphates, preference is given in particular to sodium alkyl ether sulphates based on di- or triethoxylated lauryl and myristyl alcohol. They are significantly superior to the alkyl sulphates with regard to the insensitivity towards water hardness, the ability to be thickened, the solubility at low temperature and in particular the skin and mucosa compatibility. Lauryl ether sulphate has better foam properties than myristyl ether sulphate, but is inferior to this in terms of mildness.

Alkyl ether carboxylates are types of the mildest surfactants in general, but exhibit poor foam and viscosity behaviour. They are often used in combination with alkyl ether sulphates and amphoteric surfactants.

Sulphosuccinic acid esters (sulphosuccinates) are mild and readily foaming surfactants, but on account of their poor ability to be thickened, are preferably used only together with other anionic and amphoteric surfactants and, on account of their low hydrolysis stability, are used preferably only in neutral or well buffered products.

Amidopropylbetaines have excellent skin and eye mucosa compatibility. In combination with other surfactants, their mildness can be improved synergistically. Preference is given to the use of cocamidopropylbetaine.

Amphoacetates/amphodiacetates have, as amphoteric surfactants, very good skin and mucosa compatibility and can have a conditioning effect and/or increase the care effect of supplements. Like the betaines, they are used for optimizing alkyl ether sulphate formulations. Sodium cocoamphoacetate and disodium cocoamphodiacetate are most preferred.

Alkyl polyglycosides are mild, have good universal properties, but are weakly foaming. For this reason, they are preferably used in combinations with anionic surfactants.

The cosmetic compositions according to the invention can furthermore comprise humectants.

The cosmetic composition according to the invention can comprise one or a mixture of different humectants in an amount of 0% by weight to 20% by weight, advantageously 0.1% by weight to 15% by weight, particularly advantageously 1% by weight to 10% by weight, based on the total weight of the composition according to the invention.

Particularly advantageous humectants or moisturizers within the context of the present invention are, for example, glycerol, polyglycerol, sorbitol, dimethyl isosorbide, lactic acid and/or lactates, in particular sodium lactate, butylene glycol, propylene glycol, biosaccaride gum-1, glycine soya, hydroxyethylurea, ethylhexyloxyglycerol, pyrrolidonecarboxylic acid and urea. In addition, it is especially advantageous to use polymeric “moisturizers” from the group of water-soluble and/or water-swellable and/or water-gellable polysaccharides. For example, hyaluronic acid, chitosan and/or a fucose-rich polysaccharide, which is available under the name Fucogel™ 1000 from SOLABIA S.A., are especially advantageous.

The cosmetic compositions according to the invention can also contain oils. Oils can especially be used in W/O, W/Si and O/W emulsions.

If present, the fatty phase of the composition according to the invention can comprise one non-volatile oil and/or volatile oils and waxes. The O/W composition comprises advantageously 0.01 to 45% by weight of oils, based on the total weight of the composition, and particularly advantageously 0.01 to 20% by weight of oils. The W/O or W/Si composition advantageously comprises at least 20% by weight of oils, based on the total weight of the composition.

The non-volatile oil is advantageously selected from the group of mineral, animal, vegetable or synthetic origin, polar or nonpolar oils and mixtures thereof.

The lipid phase of the cosmetic or dermatological emulsions according to the invention can advantageously be selected from the following group of substances:

mineral oils, mineral waxes, polar oils, such as triglycerides of capric acid or of caprylic acid, also natural oils, such as, for example, castor oil, fats, waxes and other natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids; alkyl benzoates; silicone oils, such as dimethylpolysiloxanes, diethylpolysiloxanes, diphenylpolysiloxanes, and mixed forms thereof.

The polar oils are advantageously selected from the group:

a) esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms,
b) esters of aromatic carboxylic acids and saturated and/or unsaturated, branched and/or unbranched alcohols of chain length from 3 to 30 carbon atoms.

Such ester oils can then advantageously be selected from the group:

isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, isotridecyl isononanoate, 2-ethylhexyl palmitate, 2-ethylhexyl laurate, 2-ethylhexyl isostearate, 2-hexyldecyl stearate, 2-octyldodecyl palmitate, 2-ethylhexyl cocoate, oleyl oleate, oleyl erucate, erucyl oleate, erucyl erucate, dicaprylyl carbonate (Cetiol® CC) and cocoglycerides (Myritol® 331), and also synthetic, semisynthetic and natural mixtures of such esters, e.g. jojoba oil.
c) alkyl benzoates C12-15-alkyl benzoate (Finsolv® TN from Finetex) or 2-phenylethyl benzoate (X-Tend® 226 from ISP)
d) lecithins and the fatty acid triglycerides, namely the triglycerol esters of saturated and/or unsaturated, branched and/or unbranched alkanecarboxylic acids of chain length from 8 to 24, in particular 12 to 18 carbon atoms. For example, the fatty acid triglycerides can be selected from the group of cocoglyceride, olive oil, sunflower oil, soybean oil, peanut oil, rapeseed oil, almond oil, palm oil, coconut oil, castor oil, wheat germ oil, grapeseed oil, safflower oil, evening primrose oil, macadamia nut oil, apricot kernel oil, avocado oil and the like.
e) dialkyl ethers and dialkyl carbonates, e.g. dicaprylyl ether (Cetiol® OE from Cognis) and/or dicaprylyl carbonate (for example Cetiol® CC from Cognis) are advantageous
f) saturated or unsaturated, branched or unbranched alcohols, such as, for example, octyldodecanol.

The non-volatile oil can likewise advantageously also be a nonpolar oil which is selected from the group of branched and unbranched hydrocarbons, in particular mineral oil, vaseline oil, paraffin oil, squalane and squalene, polyolefins, for example polydecenes, hydrogenated polyisobutenes, C13-16 isoparaffin and isohexadecane.

The nonpolar non-volatiile oil can be selected among the non-volatile silicone oils.

Of the non-volatile silicone oils, the polydimethylsiloxanes (PDMS), which are optionally phenylated, such as phenyltrimethicone, or are optionally substituted with aliphatic and/or aromatic groups or with functional groups, for example hydroxyl groups, thiol groups and/or amino groups; polysiloxanes modified with fatty acids, fatty alcohols or polyoxyalkylenes and mixtures thereof can be given.

Particularly advantageous oils are 2-ethylhexyl isostearate, octyldodecanol, isotridecyl isononanoate, isoeicosane, 2-ethylhexyl cocoate, C12-15 alkyl benzoate, caprylic/capric triglyceride, dicaprylyl ether, mineral oil, dicaprylyl carbonate, cocoglycerides, butylene glycol dicaprylate/dicaprate, hydrogenated polyisobutenes, cetaryl isononanoates, isodecyl neopentanoates, squalane, C13-16 isoparaffin.

The cosmetic composition according to the invention can also comprise a volatile oil which is selected from the group of volatile hydrocarbon oils, siliconized oils or fluorinated oils.

The volatile oil can be present in an amount of from 0 to 25% by weight, based on the total weight of the emulsion, preferably 0 to 20% by weight and even more preferably 0 to 15% by weight.

Within the context of the present specification, a volatile oil is an oil which, upon contact with the skin at room temperature and atmospheric pressure, evaporates in less than one hour.

The volatile oil is liquid at room temperature and, at room temperature and atmospheric pressure, has a vapour pressure of from 0.13 to 40 000 Pa (10⁻³ to 300 mm Hg), preferably 1.3 to 13 000 Pa (0.01 to 100 mmHg) and particularly preferably 1.3 to 1300 Pa (0.01 to 10 mmHg) and a boiling point of from 150 to 260° C. and preferably 170 to 250° C.

A hydrocarbon oil is understood as meaning an oil which is formed from carbon atoms and hydrogen atoms and optionally oxygen atoms or nitrogen atoms and contains no silicon atoms or fluorine atoms, where it may also consist of carbon atoms and hydrogen atoms; however, it can also contain ester groups, ether groups, amino groups or amide groups.

A siliconized oil is understood as meaning an oil which contains at least one silicon atom and in particular Si—O groups.

A fluorinated oil is to be understood as meaning an oil which contains at least one fluorine atom.

The volatile hydrocarbon oil according to the invention can be selected from the hydrocarbon oils with a flash point of from 40 to 102° C., preferably 40 to 55° C. and even more preferably 40 to 50° C.

For example, the volatile hydrocarbon oils are those with 8 to 16 carbon atoms and mixtures thereof, in particular branched C₈₋₁₆-alkanes, such as the isoalkanes (which are also referred to as isoparaffins) with 8 to 16 carbon atoms, isododecane, isodecane, isohexadecane and, for example, the oils which are supplied under the tradenames Isopars® or Permetyls®; and the branched C₈₋₁₆-esters, such as isohexyl neopentanoate and mixtures thereof.

The volatile hydrocarbon oils such as isododecane, isodecane and isohexadecane are particularly advantageous.

The volatile siliconized oil according to the invention can be selected from the siliconized oils with a flash point of from 40 to 102° C., preferably a flash point above 55° C. and at most 95° C. and particularly preferably in the range from 65 to 95° C.

For example, the volatile siliconized oils are straight-chain or cyclic silicone oils having 2 to 7 silicon atoms, where these silicones optionally contain alkyl or alkoxy groups having 1 to 10 carbon atoms.

The volatile siliconized oils such as octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and mixtures thereof are particularly advantageous.

The volatile fluorinated oil generally has no flash point.

For example, the volatile fluorinated oils are nonafluoroethoxybutane, nonafluoromethoxybutane, decafluoropentane, tetradecafluorohexane, dodecafluoropentane and mixtures thereof.

The composition according to the invention can also comprise a wax. Within the context of the present specification, a wax is defined as a lipophilic fatty substance which is solid at room temperature (25° C.) and exhibits a reversible solid/liquid change in state at a melting temperature between 30° C. and 200° C. Above the melting point, the wax becomes low viscosity and miscible with oils.

The wax is advantageously selected from the groups of natural waxes, such as, for example, cotton wax, carnauba wax, candelilla wax, esparto wax, Japan wax, Montan wax, sugarcane wax, beeswax, wool wax, shellac, microwaxes, ceresine, ozokerite, ouricury wax, cork fibre wax, lignite waxes, berry wax, shea butter or synthetic waxes, such as paraffin waxes, polyethylene waxes, waxes produced by Fischer-Tropsch synthesis, hydrogenated oils, fatty acid esters and glycerides which are solid at 25° C., silicone waxes and derivatives (alkyl derivatives, alkoxy derivatives, and/or esters of polymethylsiloxane) and mixtures thereof.

The waxes can be present in the form of stable dispersions of colloidal wax particles which can be prepared by known processes, for example as in “Microemulsions Theory and Practice”, L.M. Prince Ed., Academic Press (1977), pages 21-32.

Waxes may be present in amounts of from 0 to 10% by weight, based on the total weight of the composition, and preferably 0 to 5% by weight.

The cosmetic compositions according to the invention can also contain thickeners. Advantageous thickeners in particular for hair treatment applications include:
Crosslinked or uncrosslinked acrylic acid or methacrylic acid homopolymers or copolymers. These include crosslinked homopolymers of methacrylic acid or acrylic acid, copolymers of acrylic acid and/or methacrylic acid and monomers which are derived from other acrylic or vinyl monomers, such as C10-30 alkyl acrylates, C10-30-alkyl methacrylates and vinyl acetate.
Thickening polymers of natural origin, for example based on cellulose, guar gum, xanthan, scleroglucan, gellan gum, rhamsan and karaya gum, alginates, maltodextrin, starch and its derivatives, carob seed flour, hyaluronic acid.
Nonionic, anionic, cationic or amphoteric associative polymers, e.g. based on polyethylene glycols and their derivatives, or polyurethanes.
Crosslinked or uncrosslinked homopolymers or copolymers based on acrylamide or methacrylamide, such as homopolymers of 2-acrylamido-2-methylpropanesulfonic acid, copolymers of acrylamide or methacrylamide and methacryloyloxyethyltrimethylammonium chloride or copolymers of acrylamide and 2-acrylamido-2-methylpropanesulfonic acid.

Particularly advantageous thickeners are thickening polymers of natural origin, crosslinked acrylic acid or methacrylic acid homopolymers or copolymers and crosslinked copolymers of 2-acrylamido-2-methylpropanesulfonic acid.

Very particularly advantageous thickeners are xanthan gum, such as the products supplied under the names Keltrol® and Kelza® by CP Kelco or the products from RHODIA with the name Rhodopol, and guar gum, such as the products available under the name Jaguar® HP105 from RHODIA.

Very particularly advantageous thickeners are crosslinked homopolymers of methacrylic acid or acrylic acid which are commercially available from Lubrizol under the names Carbopol® 940, Carbopol® 941, Carbopol® 980, Carbopol® 981, Carbopol® ETD 2001, Carbopol® EDT 2050, Carbopol® 2984, Carbopol® 5984 and Carbopol® Ultrez 10, from 3V under the names Synthalen® K, Synthalen® L and Synthalen® MS, and from PROTEX under the names Modarez® V 1250 PX, Modarez® V2000 PX, Viscaron® A1600 PE and Viscaron® A700 PE.

Very particularly advantageous thickeners are crosslinked polymers of acrylic acid or methacrylic acid and a C₁₀₋₃₀-alkyl acrylate or C₁₀₋₃₀-alkyl methacrylate and copolymers of acrylic acid or methacrylic acid and vinylpyrrolidone. Such copolymers are commercially available, for example, from Lubrizol under the names Carbopol® 1342, Carbopol® 1382, Pemulen® TR1 or Pemulen® TR2 and from Ashland under the names Ultrathix P-100 (INCI: Acrylic Acid/VP Crosspolymer).

Very particularly advantageous thickeners are crosslinked copolymers of 2-acrylamido-2-methylpropanesulfonic acid. Such copolymers are available, for example, from Clariant under the names Aristoflex® AVC (INCI: Ammonium Acryloyldimethyltaurate/VP Copolymer).

If the thickeners are used, they are generally present in a concentration of from 0% to 2% by weight, preferably 0% to 1% by weight, based on the total weight of the composition.

Optionally, one or more preservatives may be included in the cosmetic compositions of the present invention. Examples of such preservatives comprise one or more glycerin containing compound (e.g., glycerin or ethylhexylglycerin or phenoxyethanol), benzyl alcohol, EDTA, potassium sorbate and/or grapefruit seed extract. In a preferred embodiment, the hair straightening formulations are paraben free. Details on preservatives are disclosed in US 2009/0165812. Further suitable traditional preservatives for compositions of this invention are alkyl esters of para-hydroxybenzoic acid. Other preservatives which have more recently come into use include hydantoin derivatives such as 1,3-bis(hydroxymethyl)-5,5-dimthylhydantoin, propionate salts, and a variety of quaternary ammonium compounds such as benzalkonium chloride, quaternium 15 (Dowicil 200), benzethonium chloride, and methylbenzethonium chloride. Cosmetic chemists are familiar with appropriate preservatives and routinely choose them to satisfy the preservative challenge test and to provide product stability. Suitable preservatives are benzyl alcohol, mixture of ethylhexylglycerin with benzyl alcohol, 2-bromo-2 nitropropane 1,3 diol, disodium EDTA, phenoxyethanol, mixture of phenoxyethanol and ethylhexylglycerin, methyl paraben, propyl paraben, imidazolidinyl urea (commercially available as Germall 1157), sodium dehydroacetate and benzyl alcohol. The preservatives should be selected having regard for the use of the composition and possible incompatibilities between the preservatives and other ingredients in the emulsion.

Preservatives preferably are employed in amounts ranging from about 0% to about 5%, more preferably from about 0.01% to about 2.5%, and most preferably from about 0.1% to about 1%, by weight of the composition.

The cosmetic composition according to the invention can comprise care agents, especially hair care agents. Suitable care agents are for example silicon oils and/or silicon gums, such as dialkyl- and arylsiloxanes, particularly dimethylpolysiloxane and methylphenylpolysiloxane and alkoxylated, quarternated or anionic derivatives thereof. Preferred are cyclic or linear polydialkylsiloxanes or alkoxylated and/or aminated derivatives thereof, dihydroxypolydimethylsiloxanes or polyphenylalkoxysilanes.

Further care agents can be selected from proteinhydrolysates or derivatives thereo. Proteinhydrolysates are product mixtures which are obtainable by acid, base or enzymatic catalysed degradation of proteins. proteinhydrolysates are in principle also totalhydrolysates of amino acids, amino acid derivatives, or single amino acids and their mixtures.

Suitable care agents are also vitamins, provitamines and precursors of vitamins, selected from the groups of A, B, C, E, F and H vitamins.

Further suitable care agents are also glycerol, propyleneglycol or panthenol. Plant extracts. mono- or oligosaccharides and/or lipids can also be used as care agents. Suitable care agents are also Oils, for example vegetable oils, liquid paraffinoils, ios-paraffines and synthetic carbohydrates and/or di-n-alkylethers with 12 to 36 C-atoms, esterols, such as esters from C6 to C30 fatty acids with C2 to C30 fatty alcohols, preferably monoesters on fatty acids with C2 to C24 alcohols. Examples for such esterols arelsopropylmyristat (Rilanit® IPM), Isononaic acid-C16-18-alkylester (Cetiol® SN), 2-Ethylhexylpalmitate (Cegesoft® 24), Stearic acid-2-ethylhexylester (Cetiol® 868), Cetyloleate, Glyceroltricaprylaet, Cetiol® LC, n-Butylstearate, Oleylerucate (Cetiol® J 600), Isopropylpalmitate (Rilanit® IPP), Oleyl Oleate (Cetiol®), Lauric acidhexylester (Cetiol® A), Di-n-butyladipate (Cetiol® B), Myristylmyristate (Cetiol® MM), Cetearyl Isononanoate (Cetiol® SN), Oleic aciddecylester (Cetiol® V).

Further suitable substances are dicarboxylic esters, symetric or asymmetric or cyclic ester of carbonic acid with fatty alcohols, trifattyacidesters from saturated and/or unsaturated linear or branched fatty acids with glycerol or mono- or diglycerols and their mixtures.

Especially preffered are panthenol und/oder cyclic Polydimethylsiloxanes (Cyclomethicones) or Silicon surfactants (Polyethermodified Siloxanes) of the Dimethicone Copolyol or Simethicon type. Cyclomethicones are inter alis supplied under the trade names Abil® K4 (Goldschmidt) or DC 244, DC 245 oder DC 345 from Dow Corning. Dimethicon-Copolyols are interalia supplied under the trade names DC 193 from Dow Corning or Belsil® DM 6031 from WACKER.

The composition according to the invention preferably contains 0 to 5% by weight, more preferably 0.01 to 4% by weight and most preferably 0.05 to 2% by weight, based on the total weight of the composition, of those care agents.

Cosmetic compositions for the application on nails can also contain fingernail care additives. Suitable examples are vitamins B5, E and C and derivatives thereof, and also dimethyloxobenzodioxasilanes, calcium chloride, calcium pantothenate, panthenol, proteins, ceramides, myrrhs, plant extracts, amino acid oils, for example cysteine and salts and derivatives thereof, cysteine, glutathione, biotin, urea and dimethylurea, alpha-hydroxy acids such as citric acid and ascorbic acid, UV stabilizers such as benzophenone-1, benzophenone-3, benzyl salicylate, etocrylene, drometrizole, butyl methoxydibenzoylmethane, and hardening additives such as formaldehyde and hydrolysates formed from chitin and/or keratin. Antimycotic additives are also possible. The care additives may be present in the composition according to the invention, for example, in an amount of ≥0% and ≤5% by weight, based on the total weight of the composition, and preferably of ≥0.1% and ≤3% by weight.

In order to improve the film-forming properties cosmetic compositions, especially for the application on nails can also contain plasticizers and/or coalescing agents.

Plasticizers used are advantageously plasticizers and/or plasticizing resins having a number-average molecular weight of less than 1500 g/mol, in order to achieve the desired mechanical properties. Suitable plasticizers are, for example, glycols and esters and ethers thereof, esters of acids, especially carboxylic acids, such as citrates, adipates, carbonates, tartrates, phosphates or sebacates, ethoxylated derivatives such as ethoxylated oils and/or mixtures thereof. For example, suitable plasticizers are diethylene glycol ethyl ether, diethylene glycol methyl ether, diethylene glycol n-butyl ether or diethylene glycol hexyl ether, ethylene glycol ethyl ether, ethylene glycol butyl ether, ethylene glycol hexyl ether, propylene glycol phenyl ether, propylene glycol diacetate, dipropylene glycol ethyl ether, tripropylene glycol methyl ether, diethylene glycol methyl ether, propylene glycol butyl diglycol solution, tributyl phosphate, tributoxyethyl phosphate, tricresyl phosphate, triphenyl phosphate, glycerol triacetate, butyl stearate, butyl glycolate, benzyl benzoate, butyl acetyltricinoleate, glyceryl acetyltricinoleate, dibutyl phthalate, diisobutyl phthalate, dioctyl phthalate, dimethoxyethyl phthalate, diethyl phthalate, diamyl phthalate, triethyl citrate, tributyl citrate, tributyl acetylcitrates, triethyl acetylcitrates, tri(2-ethylhexyl)acetyl-, dibutyl tartrate, triacetin, camphor, trimethylpentanyl diisobutyrate, triethylhexanoTne, sucrose benzoate, dibutyl adipate, diisobutyl adipate, diisopropyl adipate, dipropylene glycol dibenzoate and/or mixtures thereof.

Particular resins having low molecular weight may likewise be used as plasticizers and are considered to be external plasticizers since they plasticize the systems without dissolving the cellulosic film-forming resins. Advantageous plasticizers that should be mentioned are adipate polyesters, sebacate polyesters or butyl acrylate resins.

Suitable plasticizers are, for example, also the plasticizers containing carbonate groups, as described in WO2015/022438, US 2010/0158835 A1, WO 03/094870 A.

The proportion of plasticizers in the composition according to the invention is preferably in the range of ≥0% and ≤25% by weight, more preferably ≥0.5% and ≤15% by weight and most preferably ≥1% and ≤5% by weight, based on the total weight of the composition.

The preferred coalescing agents are, for example, propylene glycol ethers, for example propylene glycol n-butyl ether, propylene glycol t-butyl ether, propylene glycol n-propyl ether, propylene glycol phenyl ether, dipropylene glycol ethers, for example dipropylene glycol n-butyl ether, dipropylene glycol methyl ether, dipropylene glycol t-butyl ether, dipropylene glycol n-propyl ether, propylene glycol methyl ether acetate, propylene glycol diacetate, methyl lactate, ethyl lactate, isopropyl lactate, butyl lactates and mixtures thereof.

The proportion of coalescing agents in the composition according to the invention may, for example, be in the range of ≥0% and ≤10% by weight and preferably ≥0.1% and ≤8% by weight, based on the total weight of the composition.

Further additional active ingredients may be present in the cosmetic compositions according to the invention. These additional active agents may be selected for example from among, desquamating agents, agents for improving the skin barrier function, depigmenting agents, antioxidants, dermo-decontracting agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting the maturation of the horny envelope, NO-synthase inhibitors, peripheral benzodiazepine receptor (PBR) antagonists, agents for increasing the activity of the sebaceous glands, agents for stimulating the energy metabolism of cells, tensioning agents, lipo-restructuring agents, slimming agents, agents for promoting the cutaneous capillary circulation, calmatives and/or anti-irritants, sebo-regulators or anti-seborrhoeic agents, astringents, cicatrizing agents, anti-inflammatory agents and anti-acne agents. One skilled in this art will select the said active agent(s) as a function of the effect desired on the human body.

The formulations may also comprise one or more additional auxiliaries, e.g. acids, bases and buffers to adjust the pH value, rheology modifiers, structuring agents, fragrances, vitamines, pearlescent agents, gelling agents, trace elements, sequestering agents, antioxidants, anti-hair loss agents, antidandruff agents, ceramides, polymers, other styling polymers; further fillers, nacres, silicones or silicone derivatives, wetting agents, softeners such as glycerol, glycol and phthalic esters and ethers, UV absorbers, anticorrosive agents, neutralizing agents, antiadhesives, combining agents, antistatic agents, lustre agents, proteins and derivatives thereof, amino acids, opacifiers, stabilizers, sequestrants, complexing agents, aesthetic enhancers, fatty acids, fatty alcohols, triglycerides, botanic extracts, clarifying auxiliaries and mixtures thereof. These additives are generally present in a concentration of from about 0.001% to 25% by weight, preferably 0.01% to 15% by weight, based on the total weight of the cosmetic composition.

The cosmetic composition is preferably a product that can be applied to the human body, especially to the skin, nails or keratinous fibers, and preferably keratinous fibers.

Accordingly the cosmetic composition is a preferably a hair treatment product, especially a product for hairstyling, a skin care product, a sun care product a nail varnish or nail care product or a decorative cosmetic product for the application on human skin or keratinous fibers.

Especially preferred is the cosmetic composition according to the invention a hair treatment product, in particular a hairstyling product.

Hair treatment composition according to the invention are for example a hair setting treatment composition, a hair shaping treatment composition, a hair fixative treatment composition, a hair styling treatment composition, a hair straightening treatment composition, a hair conditioning treatment composition, a hair shine treatment composition, a hair UV protecting treatment composition, a hair softening treatment composition, a hair spray treatment composition, a permanent shaping treatment composition for hairs, a hair curl retention treatment composition, a hair film forming treatment composition.

Products for hair treatment are preferably supplied in the form of aerosols, gels, mousses, foams, lotions, waxes, pomade or creams.

Hair treating compositions according to the invention can advantageously be in the form of a pump spray or aerosol packaging. The hair setting compositions according to the invention can advantageously be foamed using a propellant gas. Accordingly, pump spray, aerosol packagings and foam dispensers based on pump spray or aerosol packaging which contain the hair setting composition according to the invention are likewise a constituent of the invention.

Preferred embodiments of aerosols are for example described in WO2014/09564 on page 27 to 28, which is herewith incorporated by reference.

The hair treatment compositions according to the invention have preferably a viscosity of ≥0.5 und ≤20000 mPas. Gels show preferably a viscosity of ≥2000 und ≤20000 mPas. Sprayable Compositions for sprays show preferably a viscosity of ≥0.5 und ≤500 mPas. Viscosities are determined according to DIN 53019 bei 23° C. with a rotation viscosimeter from Anton Paar Germany GmbH, Ostfildern, DE, at a shear rate of 10 s⁻¹ In a further embodiment of the invention is the cosmetic composition a skin care product.

A skincare product is a cosmetic composition for application to the skin, such as, in particular, to the face and/or other parts of the body. The skincare product serves in particular to protect against skin changes such as, for example, skin ageing, drying or the like. Skincare products are intended to restore the skin to or maintain the skin in its physiological normal condition. Where damage has occurred, the horny layers are aided in their natural regeneration ability, i.e. the upper horny layers are moisturized and protected. Furthermore, the permeability properties of the skin barrier should be restored and skin renewal should be aided. Additionally, a skincare product should leave behind a soft skin feel following use on the skin.

Within the context of the present invention, the skincare compositions can advantageously be present in the following forms: cream, lotion, milk, gel, oil, balm, aqueous solution.

The composition according to the invention which comprises the polyurethane described above or its aqueous dispersion should satisfy the aforementioned properties of a skincare product. Following application, the skincare composition according to the invention remains at least partially on the skin, in particular facial skin, and thus differs, for example, from cosmetic products which are removed following use on the skin, such as, for example, cosmetic face masks and cleansing products, such as soaps etc.

Within the context of the present invention, the skincare compositions are differentiated in particular according to their consistency: cream (viscous), lotion and milk (flowable), gels (semisolid), oils, and also balm and aqueous solutions (liquid). Depending on their formulation, the compositions according to the invention can be used, for example, as face cream, day or night cream, body lotion etc. It is in some instances possible that the compositions according to the invention are used as pharmaceutically active product, or comprise pharmaceutically active ingredients.

The skincare compositions may be present, for example, in the form of oil-in-water, silicone-in-water, water-in-oil, water-in-silicone, oil-in-water-in-oil, water-in-oil-in-water emulsion.

The composition can also be foamed using a propellant gas. The emulsions described above can be stabilized by an O/W, W/O or W/Si emulsifier, thickener (such as, for example, hydrodispersion) or solids (such as, for example, Pickering emulsion).

In a further embodiment of the invention the cosmetic composition is a sun care product. Within the context of the present invention, the sun protection compositions can advantageously be present in the following forms: cream, lotion, milk, gel, oil, balm, aqueous solution. The sun protection composition can also be foamed using a propellant gas.

The sun protection composition according to the invention which comprises the polyurethane described above or its aqueous dispersion should satisfy the aforementioned properties of a sun protection product. Following application, the sun protection composition according to the invention naturally remains at least partially on the skin, and thus differs, for example, from cosmetic products which are removed following use on the skin, such as, for example, cosmetic face masks and cleansing products, such as soaps etc. The sun protection composition according to the invention, furthermore, generally also does not include haircare compositions, make-up compositions, such as make-up etc., make-up lipsticks and nail varnishes or the like.

Within the context of the present invention, the sun protection compositions are differentiated in particular by their consistency: cream (viscous), lotion and milk (flowable), gels (semisolid), oils, and also liquid formulations such as, for example, spray, balm and aqueous solutions. The sun protection compositions may be present, for example, in the form of oil-in-water, water-in-oil, water-in-silicone, silicone-in-water, oil-in-water-in-oil, water-in-oil-in-water emulsion.

The emulsions described above can, for example, be stabilized by an O/W, W/O or W/Si emulsifier, thickener (such as, for example, hydrodispersion) or solids (such as, for example, Pickering emulsion).

The sun care compositions according to the invention preferably show a viscosity of ≥2 and ≤20000 mPas. Gels or Lotions preferably show a viscosity of ≥1000 and ≤20000 mPas. Sprayable compositions preferably show a viscosity of ≥2 and ≤2000 mPas.

The sun care compositions according to the invention show preferably a sun protection factor (SPF) of ≥15, determined according to the international sun protection factor test method according to COLIPA.

In a further embodiment of the invention the cosmetic composition is a nail varnish.

Nail varnish compositions in the context of the invention are especially nail varnishes, which serve to decorate the nails with colour, but also compositions for care and for protection of the nails, and likewise primers for nails to which further layers of nail varnishes may be applied.

Preferably, the nail varnish composition according to the invention further comprises organic solvents, and also plasticizers and/or special-effect constituents.

More preferably, the nail varnish composition according to the invention comprises organic solvents, rheologically modifying additives, and also plasticizers and/or special-effect constituents.

In a preferred embodiment of the invention, the nail varnish composition according to the invention comprises the polyurethane according to the invention, organic solvents special-effect constituents, plasticizers and optionally further film formers and/or additives customary in nail varnishes, such as especially rheologically modifying additives.

The nail varnish compositions according to the invention may also be systems curable by UV radiation.

The nail varnish composition according to the invention is preferably dispensed into vessels having a capacity of ≤50 ml, preferably ≤20 ml.

The invention further provides a process for producing a cosmetic coating on nails using the nail varnish compositions according to the invention, wherein the nail varnish composition is applied to nails.

Advantageously, in the process according to the invention, the nail varnish compositions according to the invention remain at least partly on the nails.

In a further embodiment of the invention the cosmetic composition is a decorative cosmetic product for the application on human skin or keratinous fibers.

The cosmetic compositions according to the invention may be also used as decorative cosmetic composition of the invention serves for the decorative, in particular colour or effect-imparting, dressing of the human skin, mucosa, semimucosa and the hair, in particular the eyelids and the eyebrows (generally not head hair). The decorative effect, i.e. colour effect or other effect (glitter effect, metallic effect etc.) is achieved by at least one effect-imparting, in particular colour- and/or effect-imparting constituent. The decorative composition according to the invention can be, for example, a face make-up (foundation), a tinted (day) cream, a blusher, a rouge, mascara, eyeliner, kohl pencil, eye shadow, lipstick, lip gloss, preferably a mascara. The decorative cosmetic compositions can be also a so-called “leave on” product which, following application, at least partially remain on the skin or the hair.

The decorative cosmetic composition according to the invention can in particular be solid, liquid or semisolid. The composition can be in the form of oil-in-water, water-in-oil, water-in-silicone oil, silicone oil-in-water, oil-in-water-in-oil, water-in-oil-in-water or solids emulsions (emulsions which are stabilized by solids, such as, for example, Pickering emulsions). The formulation according to the invention can also be foamed using a propellant gas. The formulation according to the invention can furthermore be in the form of loose powder, compact powder, mousse, sticks or in the form of the aforementioned liquid or viscous emulsions.

The cosmetic composition of the present invention can also be used in products containing fragrances or active ingredients, such as parfumes, deodorants, anti-transpirants and further applications as described for example in WO 2015/081904.

The present invention further relates to a method of a cosmetic treatment of a human being, which involves applying a cosmetic composition as before onto at least one portion of the surface of said human being. In a further preferred embodiment the method of cosmetic treatment of a human being the cosmetic composition according to the invention, following application to the surface of the human being, at least partially remains on it.

The present invention is illustrated by reference to the examples below, which are not to be understood as being limiting. Unless stated otherwise, all quantitative data, fractions and percentages are based on the weight and the total amount or on the total weight of the compositions.

### EXAMPLES

Unless designated otherwise, all of the percentages refer to the weight.

Unless noted differently, all of the analytical measurements refer to measurements at temperatures of 23° C.

The solids or solid-body contents are determined by heating a weighed sample at 125° C. to constant weight. At constant weight, the solid-body content is calculated by reweighing the sample.

Unless expressly mentioned otherwise, NCO contents were determined volumetrically in accordance with DIN-EN ISO 11909.

The control on free NCO groups was carried out by means of IR spectroscopy (band at 2260 cm⁻¹).

The stated viscosities were determined by means of rotary viscometry in accordance with DIN 53019 at 23° C. using a rotary viscometer from Anton Paar Germany GmbH, Ostfildern, Germany.

The average particle sizes (the number-average is stated) of the polyurethane dispersions were determined following dilution with deionized water by means of laser correlation spectroscopy (instrument: Malvern Zetasizer 1000, Malvern Inst. Limited).

### Example 1

289.0 of a polyester of adipic acid, hexanediol and neopentyl glycol with a number-average molecular weight of 1700 g/mol were heated to 65° C. Then, 151.4 g of Desmodur® W were added and the mixture was stirred at 125° C. until the actual NCO value had dropped below the theoretical NCO value. The molar ratio of the isocyanate groups to the hydroxyl groups in the prepolymer formation step was 3.40. The finished prepolymer was dissolved with 780 g of acetone at 50° C. and then a solution of 36.7 g of diaminosulfonate, 14.4 g of ethylene diamine and 206 g of water was metered in. The after-stirring time was 15 min. The mixture was then dispersed by adding 480 g of water. The solvent was removed by distillation in vacuo and a storage-stable dispersion was obtained, the solids content was adjusted to about 40% by weight polyurethane polymer by adding water.

### Example 2

289 g of a polyester of adipic acid, hexanediol and neopentyl glycol with a number-average molecular weight of 1700 g/mol were heated to 65° C. Then, 133.6 g of Desmodur® W (dicyclohexylmethane diisocyanate) were added and the mixture was stirred at 125° C. until the actual NCO value had dropped below the theoretical NCO value. The molar ratio of the isocyanate groups to the hydroxyl groups in the prepolymer formation step was 3.00. The finished prepolymer was dissolved with 750 g of acetone at 50° C. and then a solution of 35.0 g of diaminosulfonate (NH₂—CH₂CH₂—NH—CH₂CH₂—SO₃Na (used in 45% strength in water)), 11.3 g of ethylene diamine and 180 g of water was metered in. The after stirring time was 15 min. The mixture was then dispersed by adding 480 g of water. The solvent was removed by distillation in vacuo and a storage-stable dispersion was obtained; the solids content was adjusted to about 40% by weight polyurethane polymer by adding water.

### Example 3

297.5 of a polyester of adipic acid, hexanediol and neopentyl glycol with a number-average molecular weight of 1700 g/mol were heated to 65° C. Then, 110.0 g of Desmodur® W were added and the mixture was stirred at 125° C. until the actual NCO value had dropped below the theoretical NCO value. The molar ratio of the isocyanate groups to the hydroxyl groups in the prepolymer formation step was 2.40. The finished prepolymer was dissolved with 720 g of acetone at 50° C. and then a solution of 33.5 g of diaminosulfonate, 7.0 g of ethylene diamine and 144 g of water was metered in. The after-stirring time was 15 min. The mixture was then dispersed by adding 480 g of water. The solvent was removed by distillation in vacuo and a storage-stable dispersion was obtained, the solids content was adjusted to about 40% by weight polyurethane polymer by adding water.

### Example 4

297.5 of a polyester of adipic acid, hexanediol and neopentyl glycol with a number-average molecular weight of 1700 g/mol were heated to 65° C. Then, 100.9 g of Desmodur® W were added and the mixture was stirred at 125° C. until the actual NCO value had dropped below the theoretical NCO value. The molar ratio of the isocyanate groups to the hydroxyl groups in the prepolymer formation step was 2.20. The finished prepolymer was dissolved with 710 g of acetone at 50° C. and then a solution of 32.5 g of diaminosulfonate, 5.5 g of ethylene diamine and 130 g of water was metered in. The after-stirring time was 15 min. The mixture was then dispersed by adding 480 g of water. The solvent was removed by distillation in vacuo and a storage-stable dispersion was obtained, the solids content was adjusted to about 40% by weight polyurethane polymer by adding water.

### Example 5

318.8 of a polyester of adipic acid, hexanediol and neopentyl glycol with a number-average molecular weight of 1700 g/mol were heated to 65° C. Then, 98.3 g of Desmodur® W were added and the mixture was stirred at 125° C. until the actual NCO value had dropped below the theoretical NCO value. The molar ratio of the isocyanate groups to the hydroxyl groups in the prepolymer formation step was 2.00. The finished prepolymer was dissolved with 740 g of acetone at 50° C. and then a solution of 34.2 g of diaminosulfonate, 4.1 g of ethylene diamine and 124 g of water was metered in. The after-stirring time was 15 min. The mixture was then dispersed by adding 510 g of water. The solvent was removed by distillation in vacuo and a storage-stable dispersion was obtained, the solids content was adjusted to about 40% by weight polyurethane polymer by adding water.

### Comparative Example 1

318.8 g of a polyester of adipic acid, hexanediol and neopentyl glycol with a number-average molecular weight of 1700 g/mol were heated to 65° C. Then, 87.9 g of Desmodur® W were added and the mixture was stirred at 125° C. until the actual NCO value had dropped below the theoretical NCO value. The molar ratio of the isocyanate groups to the hydroxyl groups in the prepolymer formation step was 1.79. The finished prepolymer was dissolved with 720 g of acetone at 50° C. and then a solution of 33.9 g of diaminosulfonate, 1.6 g of ethylene diamine and 102 g of water was metered in. The after-stirring time was 15 min. The mixture was then dispersed by adding 515 g of water. The solvent was removed by distillation in vacuo and a storage-stable dispersion was obtained, the solids content was adjusted to about 40% by weight polyurethane polymer by adding water.

### Comparative Example 2

1360.0 g of a polyester of adipic acid, hexanediol and neopentyl glycol with an average molecular weight of 1700 g/mol were heated to 65° C. Then, 318.5 g of isophorone diisocyanate (IPDI) were added and the mixture was stirred at 105° C. until the NCO value was below the theoretical value. The molar ratio of the isocyanate groups to the hydroxyl groups in the prepolymer formation step was 1.79. The finished prepolymer was dissolved with 3000 g of acetone at 50° C. and then a solution of 23.4 g of isophoronediamine (IPDA), 129.6 g of diaminosulfonate and 357 g of water was metered in. The after-stirring time was 15 min. The mixture was then dispersed by adding 2900 g of water. The solvent was removed by distillation in vacuo and a storage-stable dispersion was obtained, the solids content was adjusted to 32% by weight polyurethane polymer by adding water.

### Application Tests:

### Methods:

### Bending Force Measurement Method:

Bending force measurement is a method used to quantify polymer stiffness on hair tresses. To characterize the stiffness of a polymer, peak force is measured using a miniature tensile tester (MTT175, Diastrom) with a 3-point bend fixture where the hair tress is rested on two supports that are 4.8 cm apart.

Commercially available Chinese hair (useful length: 18 cm, width: 3 cm, weight: 2.5 g) was used. The hair was subjected to a standardized washing procedure prior to use. For this, the hair was softened in water for 15 minutes and then shampooed for one minute with 0.2 ml of standard shampoo, thoroughly rinsed with warm water, blow-dried on cold and conditioned at 21±1° C. and 50±5% relative humidity. 1 g of a 2% by weight polymer mixture was applied to the tress corresponding to about 0.02 g polymer per hair tress, after drying. Each experiment was carried out with five tresses.

### High Humidity Curl Retention Measurement Method:

For the so-called “curl retention” experiments, commercially available Chinese hair (useful length: 16 cm, width: 1 cm, weight: 0.7 g, supplier: Kerling) was used. The hair was subjected to a standardized washing procedure prior to use. For this, the hair was softened in water for 15 minutes and then shampooed for one minute with 0.2 ml of standard shampoo, thoroughly rinsed with warm water, blow-dried on cold and conditioned at 21±1° C. and 50±5% relative humidity. 1 g of a 2% by weight polymer mixture was applied to the tress corresponding to about 0.12 g polymer per hair tress, after drying. Then, the hair tresses were curled onto 16 mm rollers and then conditioned at 21±1° C. and 50±5% relative humidity for at least 18 hours. The “curl retention” experiments were carried out in a special climatically controlled chamber at a relative humidity of 90±5%. The temperature in the chamber was 21±1° C. The prepared tresses were suspended simultaneously in the chamber. The length of the tresses was read off on a scale at different times. Each experiment was carried out with five tresses.

The “curl retention” was calculated according to the following formula:

CR=(I_{f}−Iₜᵢ)/(I_{f}−Iₜ)×100;

in which I_{f} was the tress length, Iₜᵢ was the original length of the curled hair tress and Iₜ was the length of the curled hair tress at time t−.

### Sensory Test Method on Flaking:

Commercially available Chinese hair (useful length: 18 cm, width: 3 cm, weight: 2.5 g) was used. The hair was subjected to a standardized washing procedure prior to use. For this, the hair was softened in water for 15 minutes and then shampooed for one minute with 0.2 ml of standard shampoo, thoroughly rinsed with warm water, blow-dried on cold and conditioned at 21±1° C. and 50±5% relative humidity. 1 g of a 2% by weight polymer mixture was applied to the tress corresponding to about 0.02 g polymer per hair tress, after drying. Then, the hair tresses were curled onto 16 mm rollers and then conditioned at 21±1° C. and 50±5% relative humidity for at least 18 hours. After drying, the hair tresses were comb through 5 times and the flaking was evaluated on the hair tress and on the comb. The curl is checked visually at the front and behind by pulling down the length of the tress. The comb is hold at different angles in order to check visually the presence of residues. Scores between 0 and 8 are linked with the amount of residue left on the comb and on the hair. The less residue there is, the highest the score is.

### Test Results:

The polymers of Examples 1 to 5 and Comparative Example 1 were subjected to the Bending Force Measurement Method and the Sensory test method on Flaking. The results are shown in table 1.

TABLE 1
Bending Force and Flaking

| | | Bending | |
|---|---|---|---|
| | Index in | Force CN | |
| Sample | Prepolymer | [gmf] | Flaking |
| Example 1 | 3.40 | 147.0 | 7 |
| Example 2 | 3.00 | 182.2 | 7 |
| Example 3 | 2.40 | 175.0 | 5.5 |
| Example 4 | 2.20 | 127.8 | 4 |
| Example 5 | 2.00 | 117.2 | 4 |
| Comparative | 1.79 | 110.0 | 2 |
| example 1 | | | |

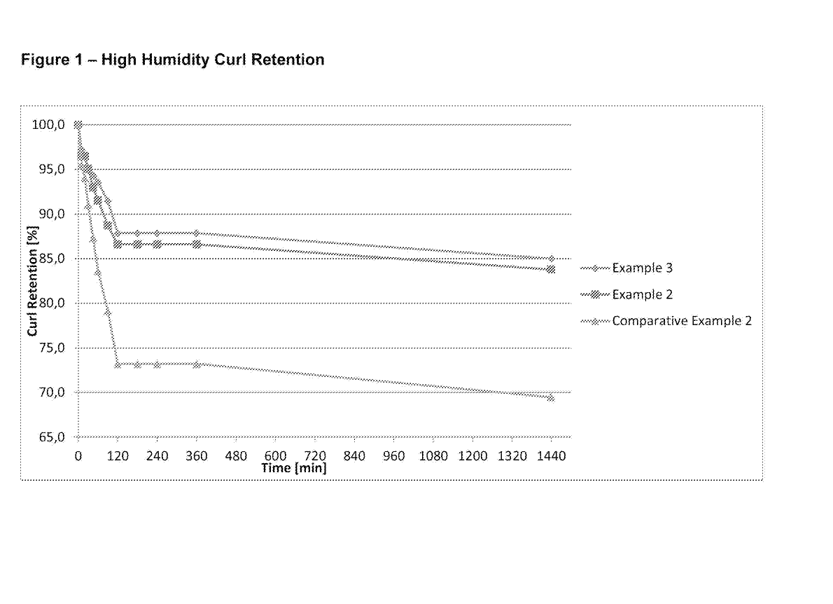

Further the polymers of Examples 2 and 3 and Comparative Example 2 were subjected to the High Humidity Curl Retention Measurement Method. The results are shown in FIG. 1.

Further examples for cosmetic compositions according to the invention are shown in the following:

### Cushion Foundation

| Ingredients [INCI name] | % by wt. |
|---|---|
| Aqua | To 100% |
| Glycerin | 5.00 |
| Dipropylen Glycol | 4.00 |
| Disodium EDTA | 0.10 |
| Preservative system | q.s. |
| Polyacrylate Crosspolymer-6 | 0.30 |
| Isonony Isononanoate | 4.00 |
| Titanium dioxide | 8.13 |
| Iron Oxide | 1.50 |
| Iron Oxide | 0.25 |
| Iron Oxide | 0.12 |
| Dipropylene Glycol | 2.00 |
| Polyurethane according to the invention | 4.00 |
| Total | 100.00 |

### Water-in-Silicone Foundation

| Ingredients [INCI name] | % by wt. |
|---|---|
| Dimethicone | 11.00 |
| PEG-9 Ricinoleate | 1.50 |
| Polytrimethylhydrosilysilicate | 3.00 |
| Propylparaben | 0.20 |
| Methylparaben | 0.20 |
| Trihydroxystearin | 2.00 |
| Magnesium Stearate | 1.00 |
| Cyclopentasiloxane (and) PEG/PPG-18/18 Dimethicone | 23.00 |
| Titanium Dioxide (and) Methicone | 8.25 |
| Iron Oxides (and) Methicone | 2.00 |
| Iron Oxides (and) Methicone | 0.59 |
| Iron Oxides (and) Methicone | 0.12 |
| Cyclopentasiloxane | 13.00 |
| Water | 21.19 |
| Diazolidinyl Urea | 0.25 |
| Magnesium Sulfate | 0.70 |
| Polyurethane according to the invention | 12.00 |
| Total | 100.00 |

### Oil-in-Water Foundation

| Ingredients [INCI name] | % by wt. |
|---|---|
| Hydrogenated Polydecene | 3.00 |
| Butylene Glycol Dicaprylate/Dicaprate | 7.50 |
| Caprylic/Capric Triglyceride | 6.00 |
| Isopropyl Palmitate | 4.00 |
| Limnanthes Alba (Meadowfoam) Seed Oil (and) | 4.00 |
| Butyrospermum Parkii (Shea) Butter Extract | |
| Sorbitan Monostearate | 1.50 |
| Glyceryl Stearate | 2.00 |
| Propylparaben | 0.15 |
| Methylparaben | 0.30 |
| BHT | 0.05 |
| Titanium Dioxide | 11.50 |
| Iron Oxides | 1.45 |
| Iron Oxides | 1.28 |
| Water | To 100 |
| Disodium EDTA | 0.05 |
| Betaine | 2.00 |
| Propylene Glycol | 2.00 |
| Glycerin | 6.00 |
| Inulin Lauryl Carbamate | 0.45 |
| Magnesium Aluminium Silicate | 0.70 |
| Microcrystalline Cellulose (and) Cellulose Gum | 0.50 |
| Polyurethane according to the invention | 12.00 |
| Water | 3.00 |
| Quaternium-15 | 0.20 |
| Total | 100.00 |

| Ingredients [INCI name] | % by wt. |
|---|---|
| Water | To 100.00 |
| Hydroxyethylcellulose | 0.5 |
| Nylon 66 | 1.5 |
| Butylene Glycol | 3.0 |
| Triethanolamine | q.s. |
| Glyceryl Stearate | 2.3 |
| Stearic Acid | 5.6 |
| Beeswax | 10.0 |
| Carnauba Wax | 4.0 |
| Dimethicone | 0.5 |
| Phenoxyethanol | 0.8 |
| Black Iron Dioxide | 8.0 |
| Polyurethane according to the invention | 16.00 |
| Total | 100.00 |

| Ingredients [INCI name] | % by wt. |
|---|---|
| Water | To 100.00 |
| Xanthan Gum | 0.80 |
| Black Iron Oxide | 10.00 |
| Laureth-4 | 1.00 |
| Propylene Glycol | 2.00 |
| PEG-10 Dimethicone | 0.50 |
| Preservative System | q.s. |
| Polyurethane according to the invention | 20.00 |
| Total | 100.00 |

### Water-Borne Nail Polish

| Ingredients [INCI name] | % by wt. |
|---|---|
| Water | To 100.00 |
| Hydroxyethylcellulose | 0.85 |
| Polysorbate 80 | 0.30 |
| Cyclomethicone | 0.10 |
| Titanium Dioxide (and) Silica | 1.60 |
| D&C Red 7 Lake | 2.00 |
| Preservative System | q.s. |
| Polyurethane according to the invention | 60.00 |
| Total | 100.00 |

### Hair Styling Gel

| Ingredients [INCI name] | % by wt. |
|---|---|
| Aqua | To 100.00 |
| Disodium EDTA | 0.05 |
| Propylene Glycol | 2.00 |
| Glycerin | 2.00 |
| Polyacrylate Crosspolymer-11 | 1.00 |
| Hydroxyethylcellulose | 0.30 |
| Preservative System | q.s. |
| Alcohol Denat. | 6.00 |
| Polyurethane according to the invention | 7.00 |
| Aminomethyl Propanol | q.s. |
| Total | 100.00 |

### Hair Styling Aerosol Spray

| Ingredients [INCI name] | % by wt. |
|---|---|
| Alcohol, Denat. | To 100.00 |
| Polyurethane according to the invention | 6.50 |
| Panthenol | 0.50 |
| Dimethylether | 50.00 |
| Total | 100.00 |

### Hair Styling Mousse

| Ingredients [INCI name] | % by wt. |
|---|---|
| Aqua | To 100.00 |
| Sodium Stearoyl Glutamate | 0.20 |
| Glycerin | 0.10 |
| Aqua (and) Hydrolyzed Keratin | 0.40 |
| Sorbitol | 1.00 |
| Lauryl Glucoside | 0.30 |
| Hydroxyethyl Cetyldimonium Phosphate | 0.20 |
| Panthenol | 0.10 |
| Tocopheryl Acetate | 0.10 |
| Aqua (and) Sodium Benzoate (and) Potassium Sorbate⁵⁾ | 0.90 |
| Fragrance | 0.05 |
| Polyurethane according to the invention | 4.00 |
| Butan | 3.00 |
| Propan | 3.00 |
| Total | 100.00 |

### Hair Styling Cream

| Ingredients [INCI name] | % by wt. |
|---|---|
| Aqua | To 100.00 |
| Xanthan Gum | 0.40 |
| Cetearyl Alcohol (and) PEG-40 Castor Oil (and) Sodium | 3.00 |
| Cetearyl Sulfate | |
| Cetearyl Alcohol | 6.00 |
| Caprylic/Capric Triglyceride | 4.00 |
| Simmondsia Chinensis Seed Oil | 4.00 |
| Persea Gratissima {Avocado} Oil | 2.00 |
| Argania Spinosa Kernel Oil | 0.50 |
| Caprylyl Methicone | 1.00 |
| Preservative System | q.s. |
| Polyurethane according to the invention | 6.50 |
| Total | 100.00 |

### Peel-Off Mask

| Ingredients [INCI name] | % by wt. |
|---|---|
| Aqua | To 100.00 |
| Acrylates/C10-30 Alkyl Acrylate Crosspolymer | 0.40 |
| Hydroxyethylcellulose | 0.40 |
| Polyurethane according to the invention | 30.00 |
| Ethanol | 5.00 |
| Dipropylene Glycol | 5.00 |
| Aminomethyl Propanol (to pH 6.0-7.0) | q.s. |
| PEG-40 Castor Oil | 0.50 |
| Propyl Glycol (and) Diazolidinyl Urea (and) Iodopropynyl | 0.80 |
| Butylcarbamate | |
| Preservative System | q.s. |
| Total | 100.00 |

### Anti-Aging Cream

| Ingredients [INCI name] | % by wt. |
|---|---|
| Aqua | To 100.00 |
| Glycerin | 2.00 |
| Propandiol | 3.00 |
| Preservative System | q.s. |
| Dipotassium Glycyrrhizanate | 0.20 |
| Coffein | 0.50 |
| Niacinamide | 1.00 |
| Sodium Stearoyl Lactylate | 0.50 |
| Sodium Hyaluronate | 0.05 |
| Sodium Acryloyldimethyltaurate/VP Copolymer | 0.60 |
| Xanthan Gum | 0.20 |
| Arachidyl Alcohol (and) Behenyl Alcohol (and) Arachidyl | 2.50 |
| Glucoside | |
| Glyceryl Stearate | 1.00 |
| Ethylhexyl Methoxycinnamate | 3.00 |
| Bis-Etylhexyloxyphenol Methoxyphenyl Triazine | 2.00 |
| Diethylamino Hydroxybenzoyl Hexyl Benzoate | 2.00 |
| Etylhexyl Triazone | 3.00 |
| Dicaprylyl Carbonate | 5.00 |
| Neopentyl Glycol Diheptanoate (and) Isododecane | 5.50 |
| Tocopheryl Acetate | 0.50 |
| Cyclopentasiloxane (and) Dimethiconol | 3.00 |
| Butylene Glycol (and) Aqua (and) Panax Ginseng Root | 2.00 |
| Extract | |
| Panthenol | 0.50 |
| Aqua (and) Sterlitza Nicolai Seed Ari Extract | 2.00 |
| Xylitylglucoside (and) Anhydroxylitol (and) Xylitol | 2.00 |
| Polyurethane according to the invention | 5.00 |
| Parfum | 0.50 |
| Total | 100.00 |

### Sun Care Lotion SPF 50

| Ingredients [INCI name] | % by wt. |
|---|---|
| Aqua | To 100.00 |
| Disodium EDTA | 0.05 |
| Glycerin | 3.00 |
| Octocrylene | 6.00 |
| Ethylhexyl Salicylate | 5.00 |
| Diethylhexyl Butamido Triazone | 5.00 |
| Bis-ethylhexyloxyphenol Methoxyphenyl Triazine | 4.00 |
| Butyl Methoxydibenzoylmethane | 5.00 |
| Diethylamino Hydroxybenzoyl Hexyl Benzoate | 1.00 |
| Propylene Glycol Dicaprylate/Dicaprate | 9.00 |
| Caprylic/Capric Triglyceride | 6.00 |
| Hydrogenated Coco-Glycerides | 1.00 |
| Methyl Glucose Sesquistearate | 2.00 |
| Cetyl Alcohol | 2.00 |
| Cetyl Palmitate | 1.00 |
| Alcohol Denat. | 8.00 |
| Polyurethane according to the invention | 7.00 |
| Total | 100.00 |

### Sun Care Lotion SPF 50

| Ingredients [INCI name] | % by wt. |
|---|---|
| Aqua | To 100.00 |
| Butylene Glycol | 2.00 |
| Tetrasodium EDTA | 0.10 |
| Arginine | 0.20 |
| Glycerin | 3.00 |
| Cetyl Hydroxyethylcellulose | 0.80 |
| Xanthan Gum | 0.40 |
| Hydrogenated Lecithin (and) Behenyl Alcohol (and) | 3.00 |
| Palmitic Acid | |
| Isoamyl Laurate | 6.00 |
| C12-C15 Alkyl Benzoate | 3.00 |
| Diisopropyl Adipate | 4.00 |
| Caprylyl Methicone | 3.00 |
| Caprylic/Capric/Myristic/Stearic Triglyceride | 2.00 |
| Bis-Diglyceryl Polyacyladipate-2 | 1.00 |
| Zinc Oxide (and) Neopentyl Glycol Diheptanoate (and) | 20.00 |
| Glyceryl Isostearate (and) Polyhydroxystearic Acid (and) | |
| Cetyl PEG-PPG-10/1 Dimethicone⁸⁾ | |
| Titanium Dioxide (and) Alumina (and) | 8.00 |
| Triethoxycaprylylsilane⁹⁾ | |
| Dimethicone (and) Dimethiconol | 2.00 |
| Alpha Bisabolol | 0.25 |
| Tocopherylacetat | 0.50 |
| Preservative System | q.s. |
| Polyurethane according to the invention | 7.50 |
| Total | 100.00 |

### Sun Care Spray SPF 50

| Ingredients [INCI name] | % by wt. |
|---|---|
| Aqua | To 100.00 |
| Glycerin | 2.00 |
| Disodium EDTA | 0.05 |
| Acrylates/C10-30 Alkyl Acrylate Crosspolymer¹⁾ | 0.10 |
| Methylene Bis-Benzotriazolyl Tetramethylbutylphenol | 4.00 |
| (and) Aqua (and) Decyl Glucoside (and) Propylene | |
| Glycol (and) Xanthan Gum | |
| Titanium Dioxide, Silica²⁾ | 2.00 |
| Ethylhexyl Salicylate | 5.00 |
| Ethylhexyl Methoxycinnamate | 6.00 |
| Butyl Methoxydibenzoylmethane | 4.90 |
| Octocrylene | 8.00 |
| C12-15 Alkyl Benzoate | 4.00 |
| Propylheptyl Caprylate | 5.00 |
| Polyurethane according to the invention | 6.00 |
| Preservative System | q.s. |
| Total | 100.00 |

1.-37. (canceled)

38. A cosmetic composition, comprising at least one water-dispersible polyurethane polymer obtainable by the reaction of at least one isocyanate-functional polyurethane prepolymer A) with at least one isocyanate-reactive component B), wherein
the at least one isocyanate-functional polyurethane prepolymer A) is obtained by the reaction of at least one polyol component A1), and
at least one polyisocyanate component A2) comprising ≥60 weight-%, based on the total weight of polyisocyanates in the prepolymer A) formation reaction, of at least one saturated cycloaliphatic polyisocyanate, and
wherein the molar ratio of the isocyanate groups to the isocyanate-reactive groups in the prepolymer A) formation reaction is at least 1.90.

39. The cosmetic composition according to claim 38, wherein the at least one polyisocyanate component A2) comprises ≥80 weight-%, more preferably 95 to 100 weight-%, based on the total weight of polyisocyanates in the prepolymer formation reaction, of at least one saturated cycloaliphatic polyisocyanate.

40. The cosmetic composition according to claim 38, wherein the prepolymer A) are obtained by reacting one or more polyols A1) selected from the group consisting of polyether polyols, polycarbonate polyols, polyether-polycarbonate polyols and polyester polyols, and one or more polyisocyanates A2) as defined before.

41. The cosmetic composition according to claim 38, wherein the polyol component A1) is selected from polyester polyols.

42. The cosmetic composition according to claim 38, wherein the polyol component A1) is selected from polyester polyols, obtained by reacting a mixture comprising adipic acid, hexanediol and neopentyl glycol.

43. The cosmetic composition according to claim 38, wherein the content of ionic and ionogenic groups in the isocyanate-functional prepolymer A) is below 15 milliequivalents per 100 g of polyurethane prepolymer A).

44. The cosmetic composition according to claim 38, wherein the water-dispersible polyurethane polymer comprises at least one hydrophilizing group, selected from the group consisting of ionic groups, ionogenic groups, non-ionic groups and non-ionogenic hydrophilizing groups.

45. The cosmetic composition according to claim 38, wherein isocyanate-reactive component B) is selected from primary and/or secondary diamines.

46. The cosmetic composition according to claim 38, wherein the isocyanate-reactive component B) comprises at least one amino-functional compound B2), which comprise at least one hydrophilizing group, selected from the group consisting of ionic groups, ionogenic groups, non-ionic groups and non-ionogenic hydrophilizing groups.

47. The cosmetic composition according to claim 38, wherein the isocyanate-reactive component B) comprises at least one amino-functional compound B2) which has ionic and/or ionogenic groups.

48. The cosmetic composition according to claim 38, wherein the isocyanate-reactive component B) comprises both, at least one amino-functional compound B2) which have ionic and/or ionogenic groups, and at least one amino-functional compound B1) which have no ionic and/or ionogenic groups.

49. The cosmetic composition according to claim 38, comprising
a) 0.05 to 65 weight-% of one or more cosmetically active ingredients, and/or one or more cosmetic auxiliary agents,
b) 5 to 99.85 weight-% one or more diluents/solvents, and
c) 0.1 to 30 weight-% at least one polyurethane as defined in claim 38,
wherein the total weight-percentages add up to 100 weight-%, based on the cosmetic composition.

50. The cosmetic composition according to claim 49, wherein the cosmetically active ingredients and/or the cosmetic auxiliary agents are selected from group consisting of film formers, propellant gas, UV absorbers, special-effect constituents, sensory additives, emulsifiers, surfactants, humectants, oils, waxes, thickeners, preservatives, care agents, especially hair care agents, fingernail care agents, plasticizers and coalescing agents.

51. The cosmetic composition according to claim 38 wherein the composition is a hairstyling composition.

52. The cosmetic composition according to claim 38 wherein the composition is a skin care product.

53. The cosmetic composition according to claim 38 wherein the composition is a sun care product.

54. A method comprising applying the cosmetic composition according to claim 38 on keratinous fibers, on human skin and on nails.

55. A method of cosmetic treatment of a human, comprising applying the cosmetic composition of claim 38 onto at least one portion of the surface of the human.

56. The method according to claim 55 wherein the cosmetic composition, following application to the surface, at least partially remains on the surface.
